(19) United States
(12) Patent Application Publication
(10) Pub. No.: US 2010/0199363 A1
(43) Pub. Date: Aug. 5, 2010

(54) ENZYMES FOR DEGRADING HERBICIDES

(76) Inventors: Carol J. Hartley, Rivett (AU); Susan J. Dorrian, Fraser (AU); Lyndall J. Briggs, Rivett (AU); Michelle R. Williams, Spence (AU); Robyn J. Russell, Wanniassa (AU); John G. Oakeshott, Wanniassa (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/299,612

(22) PCT Filed: May 11, 2007
(86) PCT No.: PCT/AU07/00640

§ 371 Date: Apr. 2, 2009

Related U.S. Application Data
(60) Provisional application No. 60/747,151, filed on May 12, 2006.

(51) Int. Cl. A01K 67/00; C12N 9/00; C07H 21/00; C12N 15/74; C12N 5/10; C12P 21/00; C07K 16/00; C12Q 1/68; A01H 5/00; A01H 1/00; C12N 1/21; C12N 1/20; A01N 63/00; A01N 37/18; G01N 33/53

(52) U.S. Cl. 800/13; 435/183; 536/23.2; 536/23.1; 435/320.1; 435/325; 435/69.1; 530/387.1; 435/6; 800/295; 800/278; 435/252.3; 435/252.1; 504/117; 504/334; 435/7.2

(57) ABSTRACT

The present invention relates to a new type of enzyme which is able to degrade amine-containing herbicides such as glyphosate and glufosinate, as well as polynucleotides encoding these enzymes. The invention also relates to transgenic plants producing these enzymes which are resistant to an amine-containing herbicide activity. In addition, the present invention provides methods of bioremediation which rely on the activity of this new type of enzyme.

### FIELD OF THE INVENTION

The present invention relates to a new type of enzyme which is able to degrade amine-containing herbicides such as glyphosate and glufosinate, as well as polynucleotides encoding these enzymes. The invention also relates to transgenic plants producing these enzymes which are resistant to an amine-containing herbicide activity. In addition, the present invention provides methods of bioremediation which rely on the activity of this new type of enzyme.

### BACKGROUND OF THE INVENTION

Organophosphonates are characterized by a stable carbon to phosphorus (C—P) bond imparting relative resistance to chemical, thermal and enzymatic degradation. Natural organophosphonates have an important role in the biogeochemical cycling of phosphorus and synthetic organophosphonates have a variety of applications in the chemical industry, most prominently as herbicides such as glyphosate (N-phosphonomethyl glycine) and phosphinothricin (Basta™)

Glyphosate (N-phosphonomethyl glycine) is a phosphonate herbicide which inhibits the 5-enolpyruvyl-3-phosphoshikimic acid synthase (EPSPS) enzyme, a component of the shikimic acid pathway. Plants utilize the shikimic acid pathway for the production of essential aromatic amino acids and vitamins, and glyphosate specifically disrupts the conversion of phosphoenolpyruvic acid and 3-phosphoshikimic acid to 5-enolpyruvyl-3-phosphoshikimic acid by EPSPS.

Once phosphonates enter the soil, microbial activity is primarily responsible for their removal, with the microbial activity divided into two principal pathways: phosphate-starvation dependent mechanisms regulated by the pho regulon whereby microbes utilize the phosphonates as a sole source of phosphorus (Wanner, 1994), and phosphate-independent mechanisms whereby bacteria utilize the phosphonates as sole source of carbon, nitrogen and phosphorus (Ternan et al., 1998a; Ternan et al., 1998b; McGrath et al., 1998). Fungal isolates capable of utilizing phosphonates (including glyphosate) as a sole source of phosphate have also been described, and it is postulated that several different pathways (possibly similar to those in bacteria) may be involved (Kryskol-Lupicka et al., 1997). The fungal isolates tested all produced AMPA as the major product from degradation of glyphosate, suggesting that a fungal enzyme similar to the bacterial GOX (WO 92/00377) is involved.

Interest has been generated in the enzymatic degradation or modification of the glyphosate herbicide, both due to concern about the environmental fate of the molecule and as an additional complementation to the systems for engineering herbicide-tolerant plants by augmenting EPSPS levels in the plant, or replacing the native EPSPS with a modified EPSPS conferring tolerance to glyphosate. Glyphosate degradation in soil has been found to be rapid and extensive, with aminomethylphosphonate (AMPA) identified as the most common product (Wackett et al. 1987), and several single bacterial cultures capable of degrading glyphosate having been described (reviewed in Malik et al., 1989).

Two routes for glyphosate metabolism in pure bacterial cultures have been characterized thus far (FIG. 1). Metabolism to AMPA and thence to complete mineralization has been described for Flavobacterium sp. (Balthazor and Hallas, 1986), Pseudomonas sp. (Jacob et al 1988) and Arthrobacter atrocyaneus (Pipke and Amrhein, 1988), whilst conversion of glyphosate to sarcosine and inorganic phosphate (Pi) by the pho regulated C—P lyase system has been described for a Pseudomonas sp. (Shinabarger and Braymer, 1986; Kishore and Jacob, 1987), Alcaligenes spec. strain GL, Streptomyces sp. (Objoska et al., 1999) and an Arthrobacter sp. (Pipke et al., 1988), and presumed for halophilic bacterium VH1 (Hayes et al., 2000) and Rhizobium meliloti 1021 (Liu et al., 1991).

Yakoleva et al. (1998) first isolated and expressed the phn operon from E. coli, encoding all the genes involved in phosphate-dependant C—P lyase activity. White and Metcalf (2004) followed up by describing two distinct operons from Pseudomomas stutzeri—one homolog of the E. coli phn operon, and another htx operon encoding hypophosphite-2oxoglutarate dioxygenase involved in phosphite metabolism. However, neither of these C—P lyase systems was able to cleave glyphosate (White and Metcalf, 2004), similarly to the substrate-specific phosphonoacetate hydrolase enzyme described by McGrath et al. (1995). Glyphosate tolerance can also be conferred by the N-acetylation of glyphosate using the glyphosate acetyltransferase (GAT) enzyme, as discovered by Castle and colleagues (Castle et al., 2004), who also utilized DNA shuffling of three different gat genes to improve the enzyme efficiency of GAT by four orders of magnitude (Castle et al., 2004; Siehl et al., 2005).

Weed control opportunities utilising such microbial herbicide-metabolizing or modifying enzymes have been discussed in detail by Padgette et al. (1996) and Vasil (1996).

Current enzymes available for degrading glyphosate, particularly when expressed in plants, do not have particularly high activity. Thus, there is the need for the identification of further enzymes which can be used to degrade herbicides such as glyphosate.

### SUMMARY OF THE INVENTION

The present inventors have identified a bacterium utilizing a previously unknown mechanism of metabolizing glyphosate. Furthermore, the inventors have isolated and characterized the novel glyphosate-degrading gene-enzyme system of this bacterium.

In one aspect, the present invention provides a substantially purified polypeptide that cleaves glyphosate to produce glycine.

In another aspect, the present invention provides a substantially purified polypeptide that cleaves the phosphonomethyl C-3 carbon to nitrogen bond of glyphosate.

In a particularly preferred embodiment, the polypeptide comprises a single amino acid chain. Thus, the polypeptide is not part of a mutlienzyme complex that requires multiple reactions to produce glycine when using glyphosate as a substrate.

In a further embodiment, the polypeptide cleaves glyphosate into glycine and oxophosphonic acid (or an ionic form thereof).

In a further particularly preferred embodiment, the polypeptide is soluble. Thus, the polypeptide is not membrane bound.

In another embodiment, substantially no glyoxylate is produced as a result of the cleavage of glyphosate.

In a further embodiment, substantially no sarcosine is produced as a result of cleavage of glyphosate.

In a further aspect, the present invention provides a substantially purified polypeptide that has a greater efficiency for cleaving glyphosate than GOX (SEQ ID NO:3).

In yet another aspect, the present invention provides a substantially purified polypeptide which has a specific activity for the cleavage of glyphosate which is greater than 550 μmolmin⁻¹mg⁻¹.

Preferably, the specific activity is greater than 600 μmolmin⁻¹mg⁻¹, more preferably greater than 700 μmolmin⁻¹mg⁻¹, and even more preferably greater than 5,000 μmolmin⁻¹mg⁻¹.

In a preferred embodiment, the specific activity of the polypeptide is determined as outlined herein in Example 3.

In a further aspect, the present invention provides a substantially purified polypeptide comprising amino acids having a sequence selected from:

i) SEQ ID NO:1, and

ii) an amino acid sequence which is at least 25% identical to i),

wherein the polypeptide cleaves an amine-containing herbicide.

In an embodiment, the polypeptide comprises a sequence provided as SEQ ID NO:8 or SEQ ID NO:9.

Examples of amine-containing herbicides include, but are not limited to, glyphosate, glufosinate, bilanafos and glyphosine. In a preferred embodiment, the amine-containing herbicide is glyphosate or glufosinate.

In a preferred embodiment, a polypeptide of the invention can be purified from an Arthrobacter species. Preferably, the Arthrobacter species is Arthrobacter spTBD.

In an embodiment, the polypeptide is fused to at least one other polypeptide.

The at least one other polypeptide may be, for example, a polypeptide that enhances the stability of a polypeptide of the present invention, or a polypeptide that assists in the purification of the fusion protein.

In a further aspect, the present invention provides an isolated polynucleotide, the polynucleotide comprising nucleotides having a sequence selected from:

i) SEQ ID NO:2,

ii) a sequence of nucleotides encoding a polypeptide of the invention,

iii) a sequence of nucleotides which is at least 25% identical to i),

iv) a sequence of nucleotides which hybridizes to i) under low stringency conditions,

v) a sequence of nucleotides complementary to i) to iv).

Preferably, the polynucleotide encodes a polypeptide that cleaves an amine-containing herbicide. More preferably, the amine-containing herbicide is glyphosate or glufosinate.

In a further embodiment, the polynucleotide encodes a polypeptide comprising a sequence provided as SEQ ID NO:8 or SEQ ID NO:9.

In a preferred embodiment, the polynucleotide comprises a sequence which hybridizes to i) under moderate stringency conditions. More preferably, the polynucleotide comprises a sequence which hybridizes to i) under stringent conditions.

In another aspect, the present invention provides a recombinant polynucleotide comprising a promoter that functions in a plant cell, operably linked to a structural DNA sequence that encodes a polypeptide of the invention, operably linked to a 3′ polyadenylation sequence that functions in the cell, wherein the promoter is heterologous with respect to the structural DNA sequence and capable of expressing the structural DNA sequence to enhance resistance of the cell to an amine-containing herbicide.

In a further aspect, the present invention provides a vector comprising a polynucleotide of the invention.

Preferably, the polynucleotide is operably linked to a promoter.

In yet a further aspect, the present invention provides a host cell comprising at least one polynucleotide of the invention, and/or at least one vector of the invention.

The host cell can be any type of cell. In one embodiment, the host cell is a plant cell.

In another aspect, the present invention provides a recombinant cell that cleaves glyphosate and produces glycine.

Preferably, the cell comprises a polynucleotide of the invention, wherein the cell does not naturally comprise said polynucleotide.

In another aspect, the present invention provides a recombinant cell comprising an introduced polypeptide that cleaves glyphosate to produce glycine.

In a further aspect, the present invention provides a recombinant cell comprising an introduced polypeptide that cleaves the phosphonomethyl C-3 carbon to nitrogen bond of glyphosate.

Preferably, the polypeptide is produced by the cell by the expression of a polynucleotide of the invention.

In another aspect, the present invention provides a process for preparing a polypeptide of the invention, the process comprising cultivating a host cell of the invention encoding said polypeptide, or a vector of the invention encoding said polypeptide, under conditions which allow expression of the polynucleotide encoding the polypeptide, and recovering the expressed polypeptide.

Also provided is a polypeptide produced using a method of the invention.

In a further aspect, the present invention provides an isolated antibody which specifically binds a polypeptide of the invention.

In yet another aspect, the present invention provides a composition comprising at least one polypeptide of the invention, at least one polynucleotide of the invention, a vector of the invention, a host cell of the invention, a recombinant cell of the invention and/or an antibody of the invention, and one or more acceptable carriers.

In a further aspect, the present invention provides a composition for cleaving an amine-containing herbicide, the composition comprising at least one polypeptide of the invention, and one or more acceptable carriers.

Preferably, the composition further comprises metal ions. In a preferred embodiment, the metal ions are divalent metal ions. More preferably, the metal ions are selected from Mg²⁺, Co²⁺, Ca²⁺, Zn²⁺, Mn²⁺, and combinations thereof. Even more preferably, the metal ions are selected from Mg²⁺, Zn²⁺, Co²⁺, and combinations thereof.

The polypeptides of the invention can be used as a selectable marker to detect a recombinant cell. Thus, also provided is the use of a polypeptide of the invention, or a polynucleotide encoding said polypeptide, as a selectable marker for detecting and/or selecting a recombinant cell.

In a further aspect, the present invention provides a method for detecting a recombinant cell, the method comprising

i) contacting a cell or a population of cells with a polynucleotide encoding a polypeptide of the invention under conditions which allow uptake of the polynucleotide by the cell(s), and

ii) selecting a recombinant cell by exposing the cells from step i), or progeny cells thereof, to an amine-containing herbicide.

Preferably, the polynucleotide comprises a first open reading frame encoding a polypeptide of the invention, and a second open reading frame not encoding a polypeptide of the invention.

In one embodiment, the second open reading frame encodes a polypeptide. In a second embodiment, the second open reading frame encodes a polynucleotide which is not translated. In both instances, it is preferred that the second open reading frame is operably linked to a suitable promoter.

Preferably, the polynucleotide which is not translated encodes a catalytic nucleic acid, a dsRNA molecule or an antisense molecule.

Examples of suitable a cell include, but are not limited to, a plant cell, bacterial cell, fungal cell or animal cell. Preferably, the cell is a plant cell.

Preferably, the amine-containing herbicide is glyphosate or glufosinate.

In a further aspect, the present invention provides a method for cleaving an amine-containing herbicide, the method comprising contacting an amine-containing herbicide with a polypeptide of the invention.

In one embodiment, the polypeptide is produced by a host cell of the invention.

Preferably, the amine-containing herbicide is glyphosate or glufosinate.

Polypeptides provided herein can be produced in plants to enhance the host plants ability to grow when exposed to an amine-containing herbicide such as glyphosate.

Thus, in yet a further aspect the present invention provides a transgenic plant comprising an exogenous polynucleotide, the polynucleotide encoding at least one polypeptide of the invention.

Preferably, the amine-containing herbicide is glyphosate or glufosinate.

In an embodiment, the polypeptide is at least produced in an aerial part of the transgenic plant.

Preferably, the polynucleotide is stably incorporated into the genome of the plant.

Also provided is a method of producing plants with enhanced resistance to an amine-containing herbicide comprising the steps of: a) inserting into the genome of a plant cell a polynucleotide comprising: a promoter that functions in plant cells to cause the production of a RNA sequence, operably linked to; a structural DNA sequence that caused the production of a RNA sequence that encodes a polypeptide of the invention, operably linked to; a 3′ non-translated region that functions in plant cells to cause the addition of polyadenyl nucleotides at the 3′ end of the RNA sequence; where the promoter is heterologous with respect to the structural DNA sequence and adapted to cause sufficient expression of the polypeptide to enhance resistance to an amine-containing herbicide of a plant cell transformed with the DNA molecule; b) obtaining a transformed plant cell; and c) regenerating from the transformed plant cell a genetically transformed plant which has increased resistance to an amine-containing herbicide.

In a further aspect, the present invention provides a transgenic plant produced using a method of the invention.

In another aspect, the present invention provides a method for cleaving an amine-containing herbicide in a sample, the method comprising exposing the sample to a transgenic plant of the invention.

Preferably, the sample is soil. Such soil can be in a field.

In another aspect, the present invention provides a transgenic non-human animal comprising an exogenous polynucleotide, the polynucleotide encoding at least one polypeptide of the invention.

In a further aspect, the present invention provides an isolated strain of Arthrobacter sp deposited under accession number V06/010960 on 11 Apr. 2006 at the National Measurement Institute, Australia.

In yet another aspect, the present invention provides a composition for cleaving an amine-containing herbicide, the composition comprising the strain of the invention and one or more acceptable carriers.

In a further aspect, the present invention provides an extract of host cell of the invention, a recombinant cell of the invention, a transgenic plant of the invention, a transgenic non-human animal of the invention, or a strain of the invention, comprising a polypeptide of the invention.

In yet another aspect, the present invention provides a composition for cleaving an amine-containing herbicide, the composition comprising the extract of the invention, and one or more acceptable carriers.

In a further aspect, the present invention provides a method for cleaving an amine-containing herbicide, the method comprising exposing an amine-containing herbicide to the strain of the invention and/or the extract of the invention.

Also provided is an isolated bacterium which produces a polypeptide of the invention.

Preferably, the bacterium is an Arthrobacter sp.

In a further aspect, the present invention provides for the use of an isolated naturally occurring bacterium which produces a polypeptide of the invention for cleaving an amine-containing herbicide.

In another aspect, the present invention provides a polymeric sponge or foam for cleaving an amine-containing herbicide, the foam or sponge comprising a polypeptide of the invention immobilized on a polymeric porous support.

In a further aspect, the present invention provides a method for cleaving an amine-containing herbicide, the method comprising exposing an amine-containing herbicide to a sponge or foam of the invention.

In another aspect, the present invention provides a product produced from a plant of the invention.

Examples of products include, but are not limited to, starch, oil, vegetables plant fibres such as cotton, malt and flour.

In a further aspect, the present invention provides a part of a plant of the invention. Examples include, but are not limited to, seeds, fruit and nuts.

The polypeptides of the present invention can be mutated, and the resulting mutants screened for altered activity such as enhanced enzymatic activity. Such mutations can be performed using any technique known in the art including, but not limited to, in vitro mutagenesis and DNA shuffling.

Thus, in a further aspect the present invention provides a method of producing a polypeptide with enhanced ability to cleave an amine-containing herbicide, the method comprising
(i) altering one or more amino acids of a first polypeptide of the invention,
(ii) determining the ability of the altered polypeptide obtained from step (i) to cleave an amine-containing herbicide, and
(iii) selecting an altered polypeptide with enhanced ability to cleave an amine-containing herbicide, when compared to the first polypeptide.

Also provided is a polypeptide produced by the method of the invention.

In yet another aspect, the present invention provides a method for screening for a microorganism capable of cleaving an amine-containing herbicide, the method comprising

i) culturing a candidate microorganism in the presence of an amine-containing herbicide as a sole nitrogen source, and

ii) determining whether the microorganism is capable of growth and/or division.

In one embodiment, the microorganism is a bacteria, fungi or protozoa.

In a preferred embodiment, the microorganism is a recombinant microorganism. Furthermore, it is preferred that a population of recombinant microorganisms are screened, wherein the recombinant microorganisms comprise a plurality of different foreign DNA molecules. Examples of such foreign DNA molecules include plasmid or cosmid genomic DNA libraries.

Preferably, the amine-containing herbicide is glyphosate.

Also provided is a microorganism isolated using a method of the invention.

In a further aspect, the present invention provides a kit comprising at least one polypeptide of the invention, at least one polynucleotide of the invention, a vector of the invention, a host cell of the invention, a recombinant cell of the invention, an antibody of the invention, a composition of the invention, at least one strain of the invention, at least one extract of the invention, at least one bacterium of the invention, at least one polymeric sponge or foam of the invention, at least one product of the invention, and/or at least one part of a plant of the invention.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. Glyphosate metabolism pathways. I. Cleavage of the C—P bond by the multi-enzyme membrane bound C—P lyase system. II. Cleavage of the C₂—N bond to produce aminomethylphosphonic acid (AMPA) and glyoxylate. III. Putative mechanism of GloxA—cleavage of the C₃—N bond to produce glycine and oxophosphonic acid.

FIG. 2. Comparison of the putative reactions catalysed by GloxA and GOX with glyphosate as a substrate (A) and HPLC reaction profiles resulting from these enzymatic reactions (B).

FIG. 3. DNA:DNA hybridizations to plasmid DNA encoding GOX or GloxA. Digested plasmid and cosmid DNA constructs were transferred to HybondN⁺ membrane and then probed with a 32^{P}-radiolabelled PCR product of the gox gene.

FIG. 4. Comparison of the activity of partially purified GloxA and GOX towards glyphosate and iminodiacetic acid.

FIG. 5. In planta expression of GloxA confers tolerance to glyphosate at five times the normal dose. To obtain the Tolerance Score, plants were visually assessed for leaf health, plant radius and flowering on Day 8 post-application of herbicide. Untreated controls in perfect health scored 7-8 under the same system.

### KEY TO THE SEQUENCE LISTING

SEQ ID NO:1—Amino acid sequence of GloxA.
SEQ ID NO:2—Nucleotide sequence encoding GloxA.
SEQ ID NO:3—Amino acid sequence of GOX.
SEQ ID NO's:4 and 5—Oligonucleotide primers.
SEQ ID NO:6—GloxA coding sequence optimized for expression in plants, includes added cloning sites at 5′ and 3′ ends as well as AACA just before start codon.
SEQ ID NO:7—GloxA coding sequence optimized for expression in E. coli.
SEQ ID NO:8—BOBL from Brevibacterium linens BL2.
SEQ ID NO:9—GloxD from Arthobacter aurescens TC1.

### DETAILED DESCRIPTION OF THE INVENTION

### General Techniques

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

### Amine-Containing Herbicides

Amine-containing herbicides include any molecule with an amine group that, when exogenously applied to a plant, has a deleterious effect on said plant. In an embodiment, the amine-containing herbicide is an organophosphonate. Examples of amine-containing herbicides include, but are not limited to, glyphosate, glufosinate, bilanafos and glyphosine.

As used herein, the term “glyphosate” refers collectively to the parent herbicide N-phosphonomethylglycine (otherwise known as glyphosate acid), to its ionic forms, to a salt or ester thereof, or to a compound which is converted to N-phosphonomethylglycine in plant tissues or which otherwise provides N-phosphonomethylglycine in ionic form (otherwise known as glyphosate ion). Glyphosate salts include, but are not restricted to, alkali metal salts, for example sodium and potassium salts; ammonium salt; C₁₋₁₆ alkylammonium, for example dimethylammonium and isopropylammonium salts; C₁₋₁₆ alkanolammonium, for example monoethanolammonium salts; C₁₋₁₆ alkylsulfonium, for example trimethylsulfonium salts; mixtures thereof and the like. The glyphosate acid molecule has three acid sites having different pKa values; accordingly mono-, di- and tribasic salts, or any mixture thereof, or salts of any intermediate level of neutralization, can be used. Glyphosate is the active ingredient of Roundup™ (Monsanto Co.). Examples of commercial formulations of glyphosate include, without restriction, those sold by Monsanto Company as ROUNDUP™, ROUNDUP™ ULTRA, ROUNDUP™ ULTRAMAX, ROUNDUP™ WEATHERMAX, ROUNDUP™ CT, ROUNDUP™ EXTRA, ROUNDUPS BIACTIVE, ROUNDUP™ BIOFORCE, RODEO™, POLARIS™, SPARK™ and ACCORD™ herbicides, all of which contain glyphosate as its isopropylammonium salt; those sold by Monsanto Company as ROUNDUP™ DRY and RIVAL™ herbicides, which contain glyphosate as its ammonium salt; that sold by Monsanto Company as ROUNDUP™ GEOFORCE, which contains glyphosate as its sodium salt; and that sold by Syngenta Crop Protection as TOUCHDOWN™ herbicide, which contains glyphosate as its trimethylsulfonium salt. Glyphosate is phytotoxic due to its inhibition of the shikimic acid pathway, which provides a precursor for the synthesis of aromatic amino acids. Glyphosate inhibits the enzyme 5-enolpyruvyl-3-phosphoshikimate synthase (EPSPS) found in plants.

As used herein, “glufosinate” refers to 2-amino-4-(hydroxymethylphosphinyl) butanoic acid and its ionic forms, esters and salts, particularly the ammonium salt. Glufosinate is a non-selective systemic herbicide which is the active ingredient of BASTA™, RELY™, FINALE™, CHALLENGE™ and LIBERTY™. Glufosinate interferes with the biosynthetic pathway of the amino acid glutamine and with ammonia detoxification.

As used herein, “bilanafos” refers to 4-hydroxy(methyl)phosphinoyl-L-homoalanyl-L-alanyl-L-alanin and its ionic forms, esters and salts.

As used herein, “glyphosine” refers to N,N-bis(p-hosphionomethyl)glycine and its ionic forms, esters and salts.

### Polypeptides

By “substantially purified polypeptide” or “purified” we mean a polypeptide that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated. As the skilled addressee will appreciate, the purified polypeptide can be a recombinantly produced polypeptide.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the invention as described herein.

As used herein, a “soluble” polypeptide does not associate with a lipid bilayer such as a cell membrane.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

As used herein a “biologically active” fragment is a portion of a polypeptide of the invention which maintains a defined activity of the full-length polypeptide, namely be able to cleave an amine-containing herbicide, especially glyphosate. Biologically active fragments can be any size as long as they maintain the defined activity. Preferably, biologically active fragments are at least 100, more preferably at least 200, and even more preferably at least 350 amino acids in length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). These DNA shuffling techniques may include genes related to those of the present invention, such as other oxidoreductases from bacteria (for example, a polynucleotide encoding SEQ ID NO:8). Products derived from mutated/altered DNA can readily be screened using techniques described herein to determine if they are able to cleave an amine-containing herbicide such as glyphosate.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides and Oligonucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. As the skilled addressee would be aware, an isolated polynucleotide can be an exogenous polynucleotide present in, for example, a transgenic organism which does not naturally comprise the polynucleotide. Furthermore, the term “polynucleotide” is used interchangeably herein with the term “nucleic acid”.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of at least about 2 kb on either end. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. The term “gene” includes a synthetic or fusion molecule encoding all or part of the proteins of the invention described herein and a complementary nucleotide sequence to any one of the above.

As used herein, the phrase “stringent conditions” refers to conditions under which a polynucleotide, probe, primer and/or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al. (supra), Current Protocols In Molecular Biology, John Wiley & Sons, N.Y. (1989), 6.3.1-6.3.6, as well as the Examples described herein. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2.×SSC, 0.01% BSA at 50° C. In another embodiment, a nucleic acid sequence that is hybridizable to the nucleic acid molecule comprising the nucleotide sequence of SEQ ID NO:2, 6 and/or 7, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art, see, e.g., Ausubel et al. (supra), and Kriegler, 1990; Gene Transfer And Expression, A Laboratory Manual, Stockton Press, NY. In yet another embodiment, a nucleic acid that is hybridizable to the nucleic acid molecule comprising the nucleotide sequences SEQ ID NO:1, 6 and/or 7, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art, see, e.g., Ausubel et al. (supra) and Kriegler, 1990, Gene Transfer And Expression, A Laboratory Manual, Stockton Press, NY, as well as the Examples provided herein.

Polynucleotides of the present invention may possess, when compared to naturally occurring molecules, one or more mutations which are deletions, insertions, or substitutions, of nucleotide residues. Mutants can be either naturally occurring (that is to say, isolated from a natural source) or synthetic (for example, by performing site-directed mutagenesis on the nucleic acid).

Oligonucleotides of the present invention can be RNA, DNA, or derivatives of either. Although the terms polynucleotide and oligonucleotide have overlapping meaning, oligonucleotide are typically relatively short single stranded molecules. The minimum size of such oligonucleotides is the size required for the formation of a stable hybrid between an oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length.

Usually, monomers of a polynucleotide or oligonucleotide are linked by phosphodiester bonds or analogs thereof to form oligonucleotides ranging in size from a relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

The present invention includes oligonucleotides that can be used as, for example, probes to identify nucleic acid molecules, or primers to produce nucleic acid molecules. Oligonucleotide of the present invention used as a probe are typically conjugated with a detectable label such as a radioisotope, an enzyme, biotin, a fluorescent molecule or a chemiluminescent molecule.

Probes and/or primers can be used to clone homologues of the polynucleotides of the invention from other species. Furthermore, hybridization techniques known in the art can also be used to screen genomic or cDNA libraries for such homologues.

### Recombinant Vectors

One embodiment of the present invention includes a recombinant vector, which comprises at least one isolated polynucleotide molecule of the present invention, inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises a polynucleotide molecule of the present invention operably linked to an expression vector. The phrase operably linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, animal, and plant cells. Vectors of the invention can also be used to produce the polypeptide in a cell-free expression system, such systems are well known in the art.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, nematode, plant or mammalian cells, such as, but not limited to, tac, lac, trp, trc, oxy-pro, omp/lpp, rrnB, bacteriophage lambda, bacteriophage T7, T71ac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

Coding sequences of the polypeptides of the invention can be optimized to maximize expression is a particular host cell using known techniques. For example, SEQ ID NO:6 provides an open reading frame encoding SEQ ID NO:1 constructed for enhanced expression in a plant cell, whereas SEQ ID NO:7 provides an open reading frame encoding SEQ ID NO:1 constructed for enhanced expression in E. coli.

### Host Cells

Another embodiment of the present invention includes a recombinant cell comprising a host cell transformed with one or more recombinant molecules of the present invention, or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides of the present invention or can be capable of producing such polypeptides after being transformed with at least one polynucleotide molecule of the present invention. Host cells of the present invention can be any cell capable of producing at least one protein of the present invention, and include bacterial, fungal (including yeast), parasite, nematode, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Particularly preferred host cells are plant cells.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

### Transgenic Plants

The term “plant” as used herein as a noun refers to whole plants, but as used as an adjective refers to any substance which is present in, obtained from, derived from, or related to a plant, such as for example, plant organs (e.g. leaves, stems, roots, flowers), single cells (e.g. pollen), seeds, plant cells and the like.

Plants contemplated for use in the practice of the present invention include both monocotyledons and dicotyledons. Target plants include, but are not limited to, the following: cereals (wheat, barley, rye, oats, rice, sorghum and related crops); beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape, mustard, poppy, olives, sunflowers, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers). Preferably, the plants are angiosperms.

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

A “transgenic plant” refers to a plant that contains a gene construct (“transgene”) not found in a wild-type plant of the same species, variety or cultivar. A “transgene” as referred to herein has the normal meaning in the art of biotechnology and includes a genetic sequence which has been produced or altered by recombinant DNA or RNA technology and which has been introduced into the plant cell. The transgene may include genetic sequences derived from a plant cell. Typically, the transgene has been introduced into the plant by human manipulation such as, for example, by transformation but any method can be used as one of skill in the art recognizes.

In a preferred embodiment, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants; see, e.g., PCT publication WO 8402913. All of these promoters have been used to create various types of plant-expressible recombinant DNA vectors.

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase promoter from wheat, the nuclear photosynthetic ST-LS1 promoter from potato, the serine/threonine kinase promoter and the glucoamylase (CHS) promoter from Arabidopsis thaliana. Also reported to be active in photosynthetically active tissues are the ribulose-1,5-bisphosphate carboxylase promoter from eastern larch (Larix laricina), the promoter for the Cab gene, Cab6, from pine, the promoter for the Cab-1 gene from wheat, the promoter for the Cab-1 gene from spinach, the promoter for the Cab 1R gene from rice, the pyruvate, orthophosphate dikinase (PPDK) promoter from Zea mays, the promoter for the tobacco Lhcb1*2 gene, the Arabidopsis thaliana Suc2 sucrose-H³⁰ symporter promoter, and the promoter for the thylakoid membrane protein genes from spinach (PsaD, PsaF, PsaE, PC, FNR, AtpC, AtpD, Cab, RbcS).

Other promoters for the chlorophyll α/β-binding proteins may also be utilized in the present invention, such as the promoters for LhcB gene and PsbP gene from white mustard (Sinapis alba). A variety of plant gene promoters that are regulated in response to environmental, hormonal, chemical, and/or developmental signals, also can be used for expression of RNA-binding protein genes in plant cells, including promoters regulated by (1) heat, (2) light (e.g., pea RbcS-3A promoter, maize RbcS promoter); (3) hormones, such as abscisic acid, (4) wounding (e.g., WunI); or (5) chemicals, such as methyl jasminate, salicylic acid, steroid hormones, alcohol, Safeners (WO 9706269), or it may also be advantageous to employ (6) organ-specific promoters.

For the purpose of expression in sink tissues of the plant, such as the tuber of the potato plant, the fruit of tomato, or the seed of soybean, canola, cotton, Zea mays, wheat, rice, and barley, it is preferred that the promoters utilized in the present invention have relatively high expression in these specific tissues. A number of promoters for genes with tuber-specific or -enhanced expression are known, including the class I patatin promoter, the promoter for the potato tuber ADPGPP genes, both the large and small subunits, the sucrose synthase promoter, the promoter for the major tuber proteins including the 22 kD protein complexes and proteinase inhibitors, the promoter for the granule bound starch synthase gene (GBSS), and other class I and II patatins promoters. Other promoters can also be used to express a protein in specific tissues, such as seeds or fruits. The promoter for β-conglycinin or other seed-specific promoters such as the napin and phaseolin promoters, can be used. A particularly preferred promoter for Zea mays endosperm expression is the promoter for the glutelin gene from rice, more particularly the Osgt-1 promoter. Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins, and the glutenins. Examples of such promoters in rice include those promoters for the ADPGPP subunits, the granule bound and other starch synthase, the branching enzymes, the debranching enzymes, sucrose synthases, and the glutelins. A particularly preferred promoter is the promoter for rice glutelin, Osgt-1 gene. Examples of such promoters for barley include those for the ADPGPP subunits, the granule bound and other starch synthase, the branching enzymes, the debranching enzymes, sucrose synthases, the hordeins, the embryo globulins, and the aleurone specific proteins.

Root specific promoters may also be used. An example of such a promoter is the promoter for the acid chitinase gene. Expression in root tissue could also be accomplished by utilizing the root specific subdomains of the CaMV 35S promoter that have been identified.

The 5′ non-translated leader sequence can be derived from the promoter selected to express the heterologous gene sequence of the polynucleotide of the present invention, and can be specifically modified if desired so as to increase translation of mRNA. For a review of optimizing expression of transgenes, see Koziel et al. (1996). An example of such an optimized leader sequence is included in SEQ ID NO:6. The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. No. 5,362,865 and U.S. Pat. No. 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. No. 4,945,050 and U.S. Pat. No. 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. An illustrative embodiment of a method for delivering DNA into Zea mays cells by acceleration is a biolistics α-particle delivery system, that can be used to propel particles coated with DNA through a screen, such as a stainless steel or Nytex screen, onto a filter surface covered with corn cells cultured in suspension. A particle delivery system suitable for use with the present invention is the helium acceleration PDS-1000/He gun is available from Bio-Rad Laboratories.

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus that express the exogenous gene product 48 hours post-bombardment often range from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. No. 5,451,513, U.S. Pat. No. 5,545,818, U.S. Pat. No. 5,877,402, U.S. Pat. No. 5,932,479, and WO 99/05265.

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions that influence the physiological state of the recipient cells and that may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. No. 5,177,010, U.S. Pat. No. 5,104,310, U.S. Pat. No. 5,004,863, U.S. Pat. No. 5,159,135). Further, the integration of the T-DNA is a relatively precise process resulting in few rearrangements. The region of DNA to be transferred is defined by the border sequences, and intervening DNA is usually inserted into the plant genome.

Modern Agrobacterium transformation vectors are capable of replication in E. coli as well as Agrobacterium, allowing for convenient manipulations as described (Klee et al., In: Plant DNA Infectious Agents, Hohn and Schell, eds., Springer-Verlag, New York, pp. 179-203 (1985)). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, In: Breeding Methods for Cultivar Development, Wilcox J. ed., American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, Canadian Patent Application No. 2,092,588, Australian Patent Application No 61781/94, Australian Patent No 667939, U.S. Pat. No. 6,100,447, International Patent Application PCT/US97/10621, U.S. Pat. No. 5,589,617, U.S. Pat. No. 6,541,257, and other methods are set out in Patent specification WO99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts.

The regenerable wheat cells are preferably from the scutellum of immature embryos, mature embryos, callus derived from these, or the meristematic tissue.

Plants expressing a polypeptide of the invention can be produced using the methods described in US 20050022261, where a polynucleotide of the invention is substituted for a nucleic acid encoding a GOX or EPSPS protein.

Glyphosate resistant wheat can be produced using the methods described in US 20040133940 where the EPSPS encoding DNA is replaced with a nucleic acid molecule encoding a polypeptide of the invention. Alternatively, glyphosate resistant wheat can be produced using a method described in US 20030154517 to introduce a gene construct encoding a polypeptide of the invention into a wheat cell.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

Plants produced using the methods described herein can be tested for resistance to a herbicide, such as glyphosate, using the procedures generally outlined in US 20050022261 or US 20060059581.

Transgenic plants of the invention may comprise further transgenes beyond those of the invention which enhance the plants tolerance/resistance to amine-containing herbicides. Examples include, the expression of bacterial EPSPS variants and plant EPSPS variants that have lower affinity for glyphosate and therefore retain their catalytic activity in the presence of glyphosate (U.S. Pat. Nos. 5,633,435, 5,094,945, 4,535,060, and 6,040,497), as well as expression of GOX which also degrades glyphosate (U.S. Pat. No. 5,776,760).

### Transgenic Hon-Human Animals

A “transgenic non-human animal” refers to an animal, other than a human, that contains a gene construct (“transgene”) not found in a wild-type animal of the same species or breed. A “transgene” as referred to herein has the normal meaning in the art of biotechnology and includes a genetic sequence which has been produced or altered by recombinant DNA or RNA technology and which has been introduced into an animal cell. The transgene may include genetic sequences derived from an animal cell. Typically, the transgene has been introduced into the animal by human manipulation such as, for example, by transformation but any method can be used as one of skill in the art recognizes.

Techniques for producing transgenic animals are well known in the art. A useful general textbook on this subject is Houdebine, Transgenic animals—Generation and Use (Harwood Academic, 1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Compositions

Compositions of the present invention may include an “acceptable carrier”. Examples of such acceptable carriers include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. The exact nature of the “acceptable carrier” will depend on the use of the composition. Considering the uses described herein, and the nature of the component of the invention in the composition, the skilled person can readily determine suitable a “acceptable carrier(s)” for a particular use.

Polypeptides, and/or expression constructs encoding therefor, can be used to treat patients, such as humans, animals and fish, which have been exposed to an amine-containing herbicide. Thus, a composition of the invention may include a “pharmaceutically acceptable carrier” to produce a “pharmaceutical composition”. Pharmaceutically acceptable carriers are well known in the art (see, for example, Remington: The Science and Practice of Pharmacy, Alfonso R. Gennaro, Mack Publishing Company, Easton, Pa., 19th Edition (1995)).

A polypeptide of the present invention can be provided in a composition which enhances the rate and/or degree of degradation of an amine-containing herbicide, or increases the stability of the polypeptide. For example, the polypeptide can be immobilized on a polyurethane matrix (Gordon et al., 1999), or encapsulated in appropriate liposomes (Petrikovics et al. 2000a and b). The polypeptide can also be incorporated into a composition comprising a foam such as those used routinely in fire-fighting (LeJeune et al., 1998).

As would be appreciated by the skilled addressee, the polypeptide of the present invention could readily be used in a sponge or foam as disclosed in WO 00/64539, the contents of which are incorporated herein in their entirety.

One embodiment of the present invention is a controlled release formulation that is capable of slowly releasing a polypeptide of the present invention into an animal, plant, animal or plant material, or the environment (including soil and water samples). As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

A preferred controlled release formulation of the present invention is capable of releasing a composition of the present invention into soil or water which is in an area sprayed with an amine-containing herbicide, particularly glyphsoate. The formulation is preferably released over a period of time ranging from about 1 to about 12 months. A preferred controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

The concentration of the polypeptide, vector, or host cell etc of the present invention that will be required to produce effective compositions for degrading an amine-containing herbicide will depend on the nature of the sample to be decontaminated, the concentration of the amine-containing herbicide in the sample, and the formulation of the composition. The effective concentration of the polypeptide, vector, or host cell etc within the composition can readily be determined experimentally, as will be understood by the skilled artisan.

### Antibodies

The invention also provides monoclonal or polyclonal antibodies to polypeptides of the invention or fragments thereof. Thus, the present invention further provides a process for the production of monoclonal or polyclonal antibodies to polypeptides of the invention.

The term “binds specifically” refers to the ability of the antibody to bind to at least one polypeptide of the present invention but not other known proteins.

As used herein, the term “epitope” refers to a region of a polypeptide of the invention which is bound by the antibody. An epitope can be administered to an animal to generate antibodies against the epitope, however, antibodies of the present invention preferably specifically bind the epitope region in the context of the entire polypeptide.

If polyclonal antibodies are desired, a selected mammal (e.g., mouse, rabbit, goat, horse, etc.) is immunised with an immunogenic polypeptide of the invention. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies of the invention may be bound to a solid support and/or packaged into kits in a suitable container along with suitable reagents, controls, instructions and the like.

In an embodiment, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, e.g., biotin. Such labeled antibodies can be used in techniques known in the art to detect polypeptides of the invention.

### Micro-Organism Deposit Details

Arthrobacter sp. TBD was deposited on 11 Apr. 2006 with the National Measurement Institute, 51-65 Clarke Street, South Melbourne, Victoria 3205, Australia under accession number V06/010960.

This deposit was made under the provisions of the Budapest Treaty on the International Recognition of the Deposit of Microorganisms for the Purpose of Patent Procedure and the Regulations thereunder. This assures maintenance of viable cultures for 30 years from the date of deposit. The organisms will be made available by the National Measurement Institute under the terms of the Budapest Treaty which assures permanent and unrestricted availability of the progeny of the culture to the public upon issuance of the pertinent patent.

The assignee of the present application has agreed that if the culture deposit should die or be lost or destroyed when cultivated under suitable conditions, it will be promptly replaced on notification with a viable specimen of the same culture. Availability of a deposited strain is not to be construed as a license to practice the invention in contravention of the rights granted under the authority of any government in accordance with its patent laws.

### EXAMPLES

### Example 1

### Isolation of Glyphosate-Degrading Bacteria

### Materials and Methods

### Chemicals

N-(phosphonomethyl)glycine (glyphosate) was obtained from ICN or Sigma. All other analytical reagents were obtained from Sigma-Aldrich.

### Media

Unless stated otherwise, minimal medium without a nitrogen source comprises M9 salts [6 g Na₂HPO₄, 3 g KH₂PO4, 1 g NaCl], trace elements including metal ions and vitamins, 200 μM MgCl₂, 200 μM CaCl₂, and 1% glucose as a carbon source.

### Isolation of Glyphosate-Degrading Bacteria

Various soil isolates and our bank of laboratory strains were tested for the ability to utilize glyphosate as a sole nitrogen source in liquid culture. Isolates were cultured overnight at appropriate temperature in rich liquid medium, either nutrient broth (Merck) or Luria broth (as in Sambrook et al., 1989), then diluted to an OD₆₀₀ₙₘ of 0.01 in minimal medium containing 0.2% (11.8 mM) glyphosate as the sole source of nitrogen. Growth was assessed by measuring OD₆₀₀ₙₘ of 2004 of culture in a flexiplate (BD Biosciences) with the Softmax Pro Platereader (Molecular Devices).

Identification of Arthrobacter sp. TBD

Genomic DNA extracted from Arthrobacter sp. TBD and universal primers were used to PCR amplify 16SrDNA and the resultant 1.35 kb product was sequenced.

### Analytical Methods

Detection of glyphosate and AMPA by HPLC analysis was performed using a modified version of the method of Tomita et al. (1991) (Column: C18 4.6 uM, 5 Å column. Mobile Phase: 0.2MK₂HPO4, 15% acetonitrile). Analytes were derivatized with tosyl chloride and detected at a wavelength of 240 nm. One ml of reaction supernatant was mixed with 0.5 mL 0.4MNaHPO₄ pH 11.0, followed by addition of 200 μL tosyl chloride solution (10 mgmL⁻¹ p-toluene sulfonylchloride [Sigma] in acetonitrile). After incubation at 50° C. for 5 minutes, 20 μL of filtered reaction product was injected onto the HPLC for analysis.

### Results

Several bacteria capable of utilizing glyphosate as sole source of nitrogen were identified, both from soil enrichment and from screening of our laboratory collection of pesticide-degrading microorganisms. The degrading bacteria included an isolate Arthrobacter sp. strain TBD which was found to be able to rapidly grow to confluence in liquid minimal medium using glyphosate as a sole source of nitrogen.

Comparison of the full length 16SrDNA sequence with the Ribosomal DataBank (Cole et al., 2005) and BLAST (McGinnis and Madden, 2004) showed that the bacterial isolate was most similar to Arthrobacter sp. strain c138 (Futamata et al., 2005), having 99.49% nucleotide identity over the 1.35 kb 16SrDNA gene sequenced.

HPLC analysis of culture supernatants revealed that Arthrobacter sp. TBD could remove 70% (8.28 mM) of the glyphosate in culture medium within 72 hours.

### Example 2

### Isolation of the Gene Responsible for Glyphosate-Degrading Activity of Arthrobacter sp. TBD

### Materials and Methods

Preparation and Screening of a Genomic DNA Library of Arthrobacter sp. TBD

Bacterial cells were cultivated to saturation with glyphosate as a sole source of nitrogen and genomic DNA was extracted from using the method of Ausubel et al. (supra). Genomic DNA was partially digested with Sau3A1, and a cosmid library created using pWEB::TNC (Epicentre) vector digested with BamHI. The library was transformed into DH10B cells, and individual plasmids were screened for the ability to confer growth using glyphosate as a sole source of nitrogen in minimal medium supplemented with solution C.

Positive colonies were selected and then the ability to confer growth with glyphosate as a sole nitrogen source confirmed in 10 mL cultures grown with shaking at 37° C. The supernatant fraction of the culture was analysed by HPLC to confirm the removal of glyphosate from the medium.

### Isolation of Gene Encoding GloxA

Cosmid pWEB::A112 was digested with Eco RI, and the 6 bands produced were subcloned into prepared vector pK18. During vector preparation, pK18 was linearised with EcoRI (NEB), and dephosphorylated with shrimp alkaline phosphatase according to manufacturer's instructions (Promega). The mini-library of Eco RI fragments from the cosmid was screened for activity as described above, and a 9 kb fragment found to confer glyphosate-degrading activity on E. coli cells. A shotgun library of sheared fragments from this construct was then sequenced to a standard of 6 times coverage (AGRF, Australia). Sequence analysis and comparison with nucleotide and protein databases were performed using Vector NTI Advance 9.0 (Informax, Invitrogen) and the NCBI BLAST (Altschul et al., 1997, 2004).

### Bioinformatic Analyses Relating to GloxA

Sequence data and predicted amino acid sequences were analysed, compiled, annotated and compared to databases using Vector NTI v.9 (Informax Inc. USA; Invitrogen, Australia) and NCBI Blast (Altshul et al, 1997). NCBI Genbank and the conserved domain database CDD were the main databases used for comparison.

### Results

### Isolation of Gene Encoding Gloxa

Screening of the cosmid library of the genomic DNA from Arthrobacter sp. TBD revealed only one cosmid which could confer the ability to grow utilizing glyphosate as a sole source of nitrogen, designated pWEB::A112. Six EcoRI restriction enzyme fragments from this construct were screened and the glyphosate-degrading activity located to a 9.5 kb EcoRI fragment, in construct pKWE1C12. Full sequence of this 9520 bp region revealed only one predicted protein with the ability to cleave glyphosate, a putative oxidoreductase encoded by ORF 9.

The glyphosate-degrading protein was designated GloxA, and sub-cloning of gloxA into an expression vector and assaying for glyphosate-degrading activity confirmed cells expressing recombinant GloxA were able to degrade glyphosate, with resting cell biocatalytic assays having glyphosate-degrading activity of 300 μmol min⁻¹ mg wet cell mass⁻¹, and crude enzyme extracts having an activity of 841 μmolmin⁻¹mg total protein (Table 2). Of importance is that glycine was not detected as a product at this stage of the analysis of GloxA, and is presumed to have been rapidly metabolized by other enzymes contained within the E. coli resting cells and crude enzyme extract preparation.

TABLE 2
The effect of metal ions and co-factors on partially
purified recombinant GloxA enzyme activity.

Catalytic Activity
(μmolmin⁻¹ mg⁻¹ wet cell mass)

| | Removal of | Production of |
|---|---|---|
| | glyphosate | glycine |
| Resting cell | 300 | — |
| biocatalytic activity | | |

Specific Activity
(μmolmin⁻¹ mg⁻¹ protein)

| | Removal of | Production of |
|---|---|---|
| | glyphosate | glycine |
| Crude enzyme extract | 841 | — |
| Partially purified enzyme | | |
| extract (PPE) | | |
| PPE + FAD + Mg²⁺ | 1590 | 548 |
| PPE − FAD + Mg²⁺ | 1450 | 562 |
| PPE + FAD − Mg²⁺ | 838 | 413 |

Crude specific activity was based on the entire protein concentration of the partially purified extract and is only an approximate value.

GloxA is a 473 amino acid protein (SEQ ID NO:1) with a molecular weight of about 52.5 kDa, a predicted isoelectric point of 5.78 and charge at pH7 of about 11.61. The coding sequence is provided as SEQ ID NO:2. Notably, the start codon is a GTG (encoding a valine residue) which is not uncommon for bacterial genes. Substitution of the start Val with a Met does not greatly alter the glyphosate degrading activity of the enzyme (Table 3).

TABLE 3
Comparison of crude enzyme activity from recombinant
proteins expressed by constructs pKWE1C12 (GTG-Val start
codon) and pETGloxA2-4 (ATG-Met start codon).

| | Removal of substrate Activity |
|---|---|
| Recombinant Construct | (μmolmin⁻¹ mg⁻¹) |
| pKWE1C12 (GTG-Val | 141 |
| start codon) | |
| pETGloxA2-4 (ATG-Met | 124 |
| start codon) | |

### Bioinformatic Analysis

The deduced amino acid sequence of GloxA (SEQ ID NO:1) shares 56% amino acid sequence identity (72% similarity) with a putative oxidoreductase from Streptomyces coelicolor A3 (Accession Number CAA20218). This amine oxidase flavoprotein is predicted to catalyze the oxidative deamination of amino acids, as well as primary and secondary amines, partially sharing substrate specificity with the monomeric sarcosine oxidase (Mörtl et al., 2004). Amine oxidase has no defined metabolic role, but shows broad substrate specificity for small amines with a low kinetic efficiency.

### Example 3

### Enzymatic Activity of GloxA

### Materials and Methods

### Enzyme Assays

Crude enzyme extracts from E. coli cells were prepared as follows. Cells were harvested and the cell pellets resuspended in 1 mL lysis buffer (25 mM Tris-Cl pH 7.5 with 1 mgml⁻¹ lysosyme) and lysed by sonication on ice (Branson sonifier, 60% duty cycle, 30 seconds on, 30 seconds off for ten repeats). The soluble fraction was collected by centrifugation at 5000 g for 5 minutes and the protein content measured using Biorad protein dye-binding assay (BIORAD). Partially purified extracts were prepared by purification of the S-tagged or HIS-tagged GloxA protein from soluble CFE using S-protein agarose (Novagen) the Talon HIS-tag Purification System (BD Biosciences), according to manufacturer's instructions. Partially pure GloxA was eluted from the column using either 3M MgCl₂ or 2.5 mM imidazole in the loading buffer. Enzyme reactions were prepared containing 500 μL crude enzyme extract or 250 μL partially purified enzyme extract (˜10 μg protein lysate) in 25 mM Tris-Cl pH7.5, 1 mM FAD, 0.1 mM MgCl2. After preincubation at 37° C. for 2 minutes, 1 mM glyphosate was added to the reaction, which was then incubated with shaking for a further 10-60 minutes. After the required reaction time, reactions were derivatized with tosyl chloride and analysed by HPLC as described above. All enzyme assays were performed in triplicate and repeated at least twice to confirm data. For determination of apparent kinetic parameters, substrate concentrations were varied over a suitable range, and the data fitted to a sum of exponential equations using Kaleidagraph (Synergy Software) or Hyper 1.1 (J. S. Easterby).

### Results

Contrary to most previous reports in the literature, we detected no production of AMPA from glyphosate by GloxA. Glycine was the most abundant product detected from reaction with glyphosate of both crude extracts and partially purified GloxA protein. From this and the homology of the predicted amino acid sequence of GloxA to other oxidoreductase flavoproteins, we conclude that GloxA is likely using an oxidation-reduction mechanism to cleave the phosphonomethyl C-3 carbon to nitrogen bond of glyphosate to yield glycine and oxophosphonic acid. Oxophosphonic acid is not detectable in the reaction supernatants but would be expected to be rapidly oxidised under aqueous conditions.

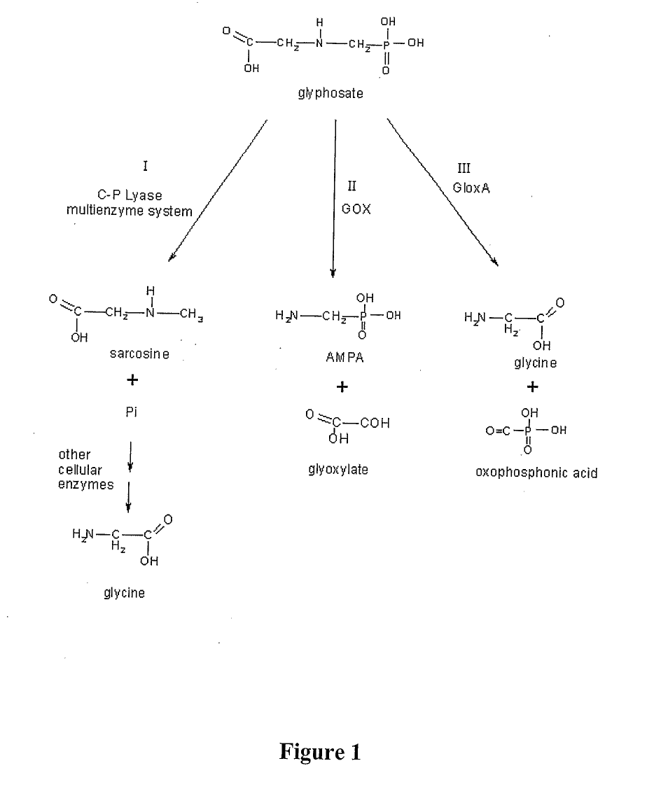

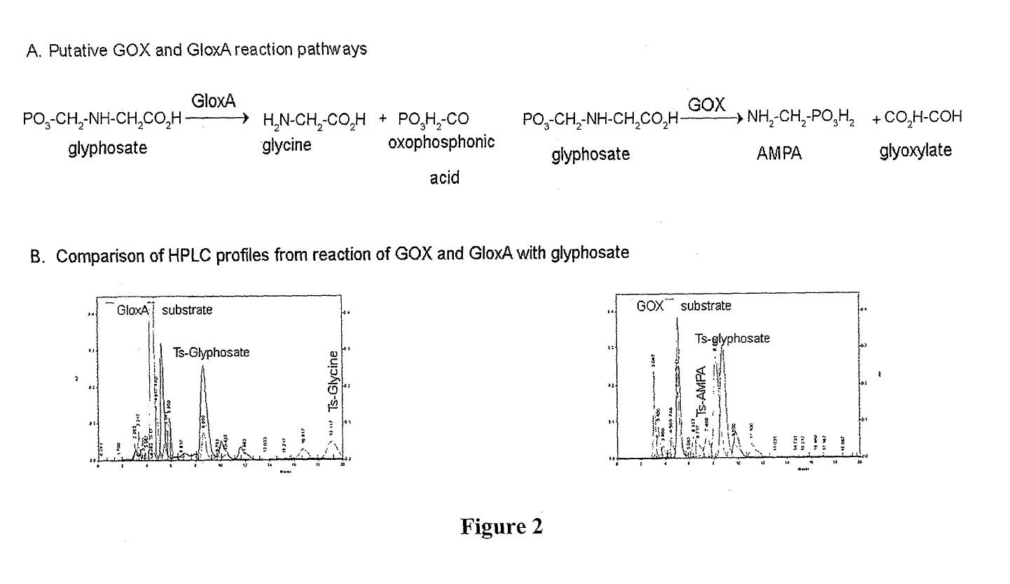

Glycine was also the end-product of cleavage of glyphosate found in Pseudomonas sp. strain PG2982 as identified by solid state NMR analysis of bacterial growth cultures (Kent-Moore et al., 1983; Jacob et al., 1985; Fitzgibbon and Braymer, 1990) and Pseudomonas sp. strain LBr (Jacob et al., 1988). Arthrobacter sp. strain GLP-10 was also reported to produce glycine from glyphosate, but this derives from cleavage of the C—P bond in glyphosate by the C—P lyase multienzyme complex, to produce sarcosine and subsequent conversion to glycine (Kishore and Jacob, 1987) (see FIG. 1). The C—P lyase reaction is catalysed by a complex membrane-bound protein system involving at least 4 different proteins (Metcalf and Wanner, 1993), none of which shares amino acid similarity with GloxA. Thus the glyphosate to glycine cleavage activity of GloxA in a cell-free manner represents a novel mechanism for the cleavage of glyphosate (FIG. 2).

The exact reaction conditions for maximal GloxA activity appears to involve metal ions and possibly the use of FAD as a co-factor, based on the relative specific activity of GloxA when in the absence of these two components (Table 2).

The substrate range of partially purified recombinant GloxA enzyme is provided in Table 4. Notably, GloxA is also able to cleave glufosinate which is a herbicide found in Basta™.

TABLE 4
Substrate range of partially purified
recombinant GloxA enzyme activity.

| | Removal of substrate | |
|---|---|---|
| | Specific Activity | Relative Activity |
| Substrate | (μmolmin⁻¹ mg⁻¹) | (%) |
| glyphosate (RoundUp, has | 841 | 100 |
| 478 g L⁻¹ of acid) | | |
| AMPA | 602 | 71 |
| iminodiacetic acid | 5013 | 598 |
| glufosinate ammonium (Basta ™ | 71 | 8.5 |
| has 200 g L⁻¹) | | |
| sarcosine | | |
| glycine | | |
| phosphinothricin | | |

Table 5 provides a comparison of the enzymatic activity of GloxA on glyphosate and glufosinate compared to some other known enzymes which degrade these compounds. Under the conditions utilized GloxA had superior glyphosate degrading activity when compared to the known enzymes.

TABLE 5
Comparison of the apparent kinetic parameters of GloxA using herbicide substrates
with other enzymes known to confer herbicide tolerance when expressed in plants.

| | Enzyme | | | | | | |
|---|---|---|---|---|---|---|---|
| | | Native | Best | Native | Best | | |
| | PAT/bar¹ | GAT² | evGAT^{§2} | GOX³ | evGOX^{§3} | Native GloxA | |
| herbicide | glufosinate | glyphosate | glyphosate | glyphosate | glyphosate | glyphosate | glufosinat [symbol] |
| K_{cat} (min⁻¹) | 470 | 5-8 | 1480 | 40 | 614 | 10798 | 9536 |
| K_{M} (mM) | 0.023 | 1-1.7 | 0.05 | 25-28 | 2.6 | 0.056 | 1.2 |
| K_{cat}/K_{M} | 20400 | 4 | 29600 | 1.6 | 236 | 192821 | 7946 |

Key: ^{§}evGAT/GOX- evolved GAT (shuffling)/GOX (SDM).
¹Blair-Kerth et al. (2001)
²Siehl et al. (2005)
³WO 92/00377. [symbol]
indicates data missing or illegible when filed

Table 2 above shows that of the conditions tested the presence of Mg²⁺ and FAD resulted in the highest activity. Further divalent metal ions were tested (Table 6) with enzymatic activity being observed in the absence of metal ions or FAD. The addition of Co²⁺ to the reaction resulted in maximal activity when compared to the other conditions tested.

TABLE 6
Effect of metal ions on partially purified GloxA enzyme activity.

| | | | Glyphosate Removal |
|---|---|---|---|
| | | | μmol/min/mg |
| A | Std (Mg²⁺) | | 856 |
| B | No FAD | | 520 |
| C | No Metals | (+EDTA) | 432 |
| D | Zn²⁺ | | 624 |
| E | Co²⁺ | | 1076 |

### Example 4

### Comparison of GloxA with GOX

### Materials and Methods

### DNA:DNA Hybridizations

Genomic DNA, plasmid and cosmid constructs were completely digested with appropriate restriction enzymes, and transferred to HybondN⁺ nylon membrane (Amersham, now GE Biosciences) using alkali transfer as described by the manufacturer.

A radiolabelled probe was generated by asymmetric PCR using 30 ng template DNA (pLSGOX) in a reaction containing 4 μL 5× Expand HiFidelity™(Roche) Buffer; 12.5 pmoles antisense primer (CGOX1B; ATGGCTGAGAACCACAAAAAAGTAG) (SEQ ID NO:4); 0.1 pmoles sense primer (CGOX2B; TTAACTTGCCGGACCCGTTTGCTTG) (SEQ ID NO:5); 200 μM each of, dTTP, dCTP, aGTP; 5 μM dATP; 0.33 μM α-³²P-dATP (Amersham) and 2U of Expand HiFidelity DNA polymerase (Roche). The reaction was cycled at 94° C. for 3 minutes, followed by 30 cycles of (94° C. for 45 secs, 48° C. for 45 secs, 72° C. for 30 secs), with a final extension of 72° C. for 5 minutes. The resultant radiolabeled PCR product was purified using QIAQuick DNA purification column (Qiagen), and eluted in 30 μL sterile water. Membranes were prehybridised in a solution containing 6×SSC, 5×Denhardt's solution, 0.1% sodium pyrophosphate (NaPPi), 0.5% SDS incubated at 65° C. (in a Hybaid oven) for 2 hours before adding 5 μL (approx. 50 μCi) of radiolabelled probe to the hybridization bottle. The hybridization was then allowed to proceed overnight (18 hrs) at 65° C. (stringent conditions) or 42° C. (low stringency conditions). Washes of varying stringency were performed as described by Ausubel et al (supra).

### GOX Expression

For the purposes of direct comparison between the enzymes, a synthetic construct was made encoding the GOX gene based on SEQ ID NO:17 from U.S. Pat. No. 5,776,760. The synthetic construct was subcloned from pLSGOX (custom manufactured by Topgene, Canada) into the Eco RI site of expression vector pET29a (Novagen) to produce the enzyme with an N-terminal S-tag. Soluble protein expression of GOX was optimized under varying temperature and IPTG conditions, and GOX protein expression detected by Coomasie-stained SDS-PAGE analysis and GOX enzymatic activity assayed as described above for GloxA, with HPLC analysis of activity to detect the removal of glyphosate and production of AMPA according to analytical methods above.

### Results

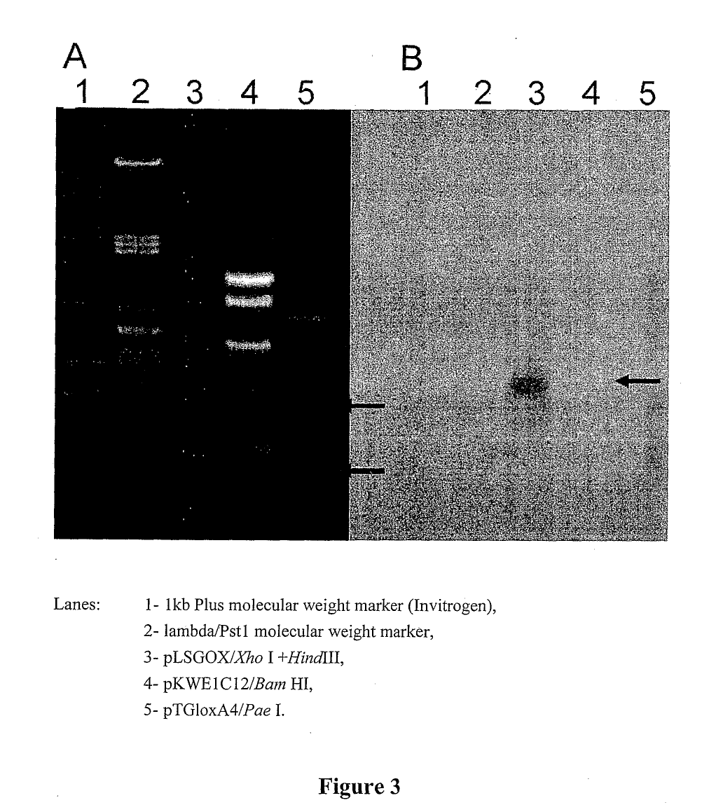

Prior to obtaining full gene sequence for the glyphosate degrading constructs described above, the homology of the isolated DNA to the gox gene was tested by DNA:DNA hybridization. The resultant southern blots illustrated no specific detectable binding between the DNA constructs and the gox gene under either standard or non-stringent conditions (FIG. 3).

Glyphosate oxidoreductase (GOX) is the only other enzyme reported in the literature which can cleave glyphosate in a single enzymatic step, producing AMPA from glyphosate purportedly by re-oxidising reduced flavin to break the C2-carbon to nitrogen bond of glyphosate to produce AMPA and glyoxylate (WO 92/00377; U.S. Pat. No. 5,776,760). The specific activity of GOX variant v.247 was described in U.S. Pat. No. 5,776,760 as 3-4 fold higher than the 15 nmolmin⁻mg⁻¹ of wildtype GOX, representing an approximate activity of 60 nmolmin⁻¹mg⁻¹. Our initial crude enzyme activity data for GloxA suggested that GloxA had much higher specific activity than GOX (Table 3, 841 μmolmin⁻¹mg⁻¹).

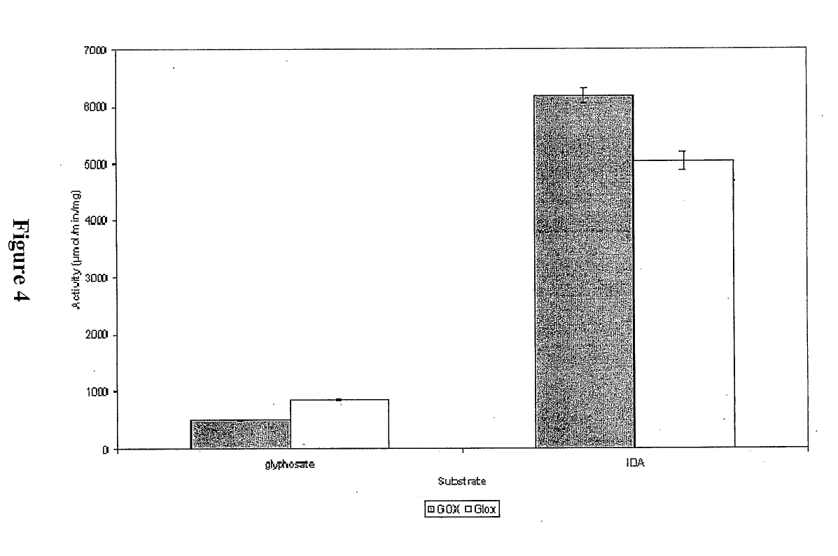

Direct comparison was necessary to confirm this, so we prepared crude extracts of a synthetic GOX construct in the same way as our GloxA variant, and assayed the two enzymes side-by-side. Under these conditions, the specific activity of GloxA towards glyphosate was almost two fold higher than GOX (FIG. 4) but similar to GOX towards iminodiacetic acid (0.9 times less, FIG. 4). The differences in activity between the data of WO 92/00377 and ourselves probably relate to several factors: only partial purification of the enzymes in our case, and differences in the expression systems and conditions used.

Both enzymes exhibited significantly higher activity towards iminodiacetic acid, and both produced glycine and glyoxylate. This is still consistent with a C2-carbon to nitrogen bond cleavage preference for GOX, and a C3-carbon to nitrogen bond cleavage preference for GloxA, as both cleavage reactions would produce glyoxylate and glycine from iminodiacetic acid (refer to FIG. 2).

### Example 5

### Comparison of GloxA with Other Homologous Proteins

### Materials and Methods

The GloxA nucleotide and amino acid sequences were compared to the non-redundant nucleotide and protein NCBI databases using BLASTN and BLASTP (Altschul et al., 1997), respectively. Selected putative GloxA-like homologous proteins were then expressed and assayed for glyphosate degradative activity. The nucleotide coding sequences of selected putative homologues were commercially synthesised (Geneart, GmBH), cloned and expressed using the Champion pET200D/TOPO. Expression System (Invitrogen), according to manufacturer's instructions. Recombinant protein expression was verified and quantitated by Coomasie-stained SDS-PAGE analysis and spot-densitometry using AlphaImager 2200 visualization and documentation system (Alpha Innotech).

Enzymatic assays to assess glyphsate degradation were, performed using soluble cell-free protein extracts (partially purified protein) in a 1 ml volume containing 100 μg total cell-free protein (˜10 μg enzyme extract), 1 mM MgCl₂, 1 mM glycine in 20 mM Tris-Cl pH 7.2. Negative controls included cell-free extracts from E. coli BL21 Star both containing no expression vector and containing pET200D/TOPO without an insert.

### Results

GloxA is previously undescribed, but is most similar to a putative oxidoreductase protein recently predicted from the complete genome annotation of Arthrobacter aurescens TC1 (86% amino acid identity; NCBI accession no. CP000474.1), and also shares a putative conserved protein domain with the DadA family of amine oxidase proteins (CDD COG0665).

Glox A shares protein sequence identity with several other proteins in the non-redundant protein databases. The homologues include a putative glycine oxidase protein predicted from the annotation of the genome of Brevibacterium linens BL2 (NCBI Accession No. ZP_{—}00381186) which we have demonstrated is also able to cleave glyphosate to produce glycine (Table 7).

TABLE 7
Comparison of GloxA with proteins having significant amino acid identity to GloxA.

| | | NCBI | % amino acid | Partially Purified Activity |
|---|---|---|---|---|
| | | Accession No./ | sequence identity | [glyphosate degradation] |
| Designation | Source | Refererence | to GloxA | (μmol/min/mg) |
| GloxA | Arthrobacter | This study | 100 | 841 |
| | sp. TBD | (SEQ ID NO: 1) | | |
| Glox D | Arthrobacter | ABM06986 | 86 | nd |
| | aurescens TC1 | (SEQ ID NO: 9) | | |
| GOBL | Brevibacterium | ZP_00381186 | 60 | 480 |
| | linens BL2 | (SEQ ID NO: 8) | | |
| IdaA | EDTA-degrading | Liu et al., 2001 | 28 | 0 |
| | bacterium BNC1 | | | |

### Example 6

### Expression of GloxA in Arabidopsis

A DNA encoding GloxA which was optimized for plant expression (SEQ ID NO:6). The sequence provided in SEQ ID NO:6 includes added cloning sites at 5′ and, 3′ ends as well as AACA just before start codon. The coding sequences spans nucleotides 16 to 1432 of SEQ ID NO:6. The DNA encoding GloxA was cloned into the Agrobacterium transfer vector, p277 (obtained from CSIRO Plant Industry, Canberra, Australia). This vector was constructed by inserting the NotI fragment from pART7 into pART27 (Gleave, 1992). The p277 vector contains the CaMV 35S promoter and OCS terminator for plant expression, markers for antibiotic selection, and the sequences required for plant transformation. The construct was synthesised by PCR and directionally cloned into the p277 transfer plasmid.

Transformation of the Agrobacterium strain GV3101 was achieved using the triparental mating method. This involves co-streaking cultures of A. tumefasciens GV3101, E. coli carrying a helper plasmid, RK2013, and E. coli carrying the desired recombinant p277 plasmid onto a non-selective LB plate. Overnight incubation at 28° C. results in a mixed culture which was collected and dilution streaked onto LB plates which selected for A. tumefasciens GV3101 carrying the p277 recombinant plasmid.

Arabidopsis plants were cultured by standard methods at 23° C. with an 18 hr light period per day. Transformation of Arabidopsis plants was carried out by floral dipping. Plants are grown to an age, 3-5 weeks, where there were many flower stems presenting flowers at various stages of development. An overnight culture of transformed A. tumefasciens GV3101 is pelleted and resuspended in 5% sucrose containing the wetting agent Silwet-77. Flowers were dipped into the bacterial suspension and thoroughly wetted by using a sweeping motion. The plants were wrapped in plastic film and left overnight on a bench top at room temperature, before being unwrapped and placed back into a plant growth cabinet maintained at 21° C. The dipping was repeated 1-2 weeks later to increase the number of transformed seeds. The seeds were collected 3-4 weeks after dipping, dried in seed envelopes for the appropriate length of time for each ecotype, then sterilised and germinated on Noble agar plates containing selective antibiotics and an antifungal agent.

Positive transformants were transplanted into Arasystem pots (Betatech), grown to maturity inside Aracon system sleeves and the seeds carefully collected. Transformed Arabidopsis plants (T1 generation) were screened by PCR and reverse-transcriptase PCR (RT-PCR) to confirm the presence and expression of the recombinant gene. Genomic DNA was extracted from the leaves of plants transformed with the construct using the Extract-N-Amp Plant PCR and Extract-N-Amp Reagent kits (Sigma). PCR on the extracts was performed using primers specific to the GloxA encoding sequence. For RT-PCR, about 8 plants transformed with the construct were randomly selected for analysis. Leaves from these plants are snap frozen and ground in liquid nitrogen using a mortar and pestle. RNA is isolated using the RNeasy Plant kit (Qiagen). cDNA is prepared from the RNA using the iScript cDNA Synthesis kit (Bio-Rad). PCR was performed using 1 μl of cDNA, recombinant Taq polymerase (Invitrogen), an annealing temperature of 54° C., and GloxA specific primers. 3 μl of each 25 μl PCR reaction is visualised on a 1.2% agarose gel. Quantitative PCR was performed using the Applied Biosystems 7000 Real-Time PCR system, with an Arabidopsis house-keeping gene araPTB (TAIR accession number AT3G01150) as a reference gene. Selected kanamycin-resistant T2 and T3 plants expressed GloxA mRNA at levels ranging from 30 to 100 times greater than the reference gene (Table 8).

TABLE 8
Quantitative RT-PCR analysis of GloxA
expression in Arabidopsis plants.

| | dCt | 2^{dCt} |
|---|---|---|
| Sample Name | (GloxArt − araPTB) | (fold difference) |
| L-er (wildtype | −5.03 ± 0.144 | 32.7x less than PTB |
| Arabidopsis) | | |
| GloxA T2 #3 | 5.86 ± 0.142 | 58.1x more than PTB |
| GloxA T2 #4 | 5.50 ± 0.175 | 45.3x more than PTB |
| GloxA T2 #5 | 5.64 ± 0.082 | 49.9x more than PTB |
| GloxA T2 #6 | 6.29 ± 0.145 | 78.2x more than PTB |
| GloxA T2 #1 | 6.81 ± 0.124 | 112.2x more than PTB |
| GloxA T2 #8 | 5.94 ± 0.184 | 61.4x more than PTB |
| GloxA T3 #2e | 1.77 ± 0.136 | 3.4x more than PTB |

*Key: Ct—threshold cycle; dCT—difference in threshold cycle (target GloxA − reference araPTB).

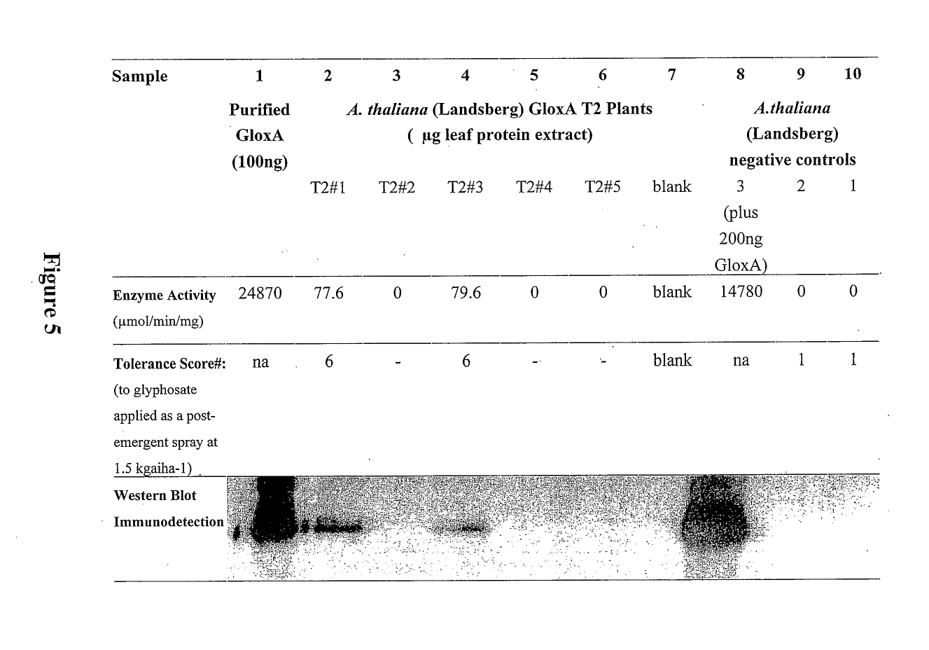

T1 seedlings can be transplanted and cultivated for seed through two generations to eventually isolate the homozygous T3 seeds. T2 and T3 plants were then screened for increased resistance to glyphosate, essentially using the methods described by Jander et al. (2003), but with A. thaliana var. Landsberg, not Columbia, and using treatment doses ranging from 2.5-25 kg a.i. ha⁻¹. The scores given in FIG. 5 are for a dose of 5× the expected I₁₀₀ dose (12.5 kga.i.ha⁻¹)

Leaves from transgenic plants from stages T1-T3 can also be analysed by extraction of total plant protein (e.g. using Pierce P-PER Plant Protein Extraction Kit) and assessment of GloxA protein expression within the plant cells using both Western Blot antibody detection systems and enzyme extract assays. For Western Blot analysis plant protein extracts were first diluted ten-fold in 20 mM Tris-Cl pH 7.2 and then quantified by Biorad Protein Dye (Biorad). Equivalent amounts of plant protein were loaded into each well of a 10% SDS-polyacrylamide gel and separated by electrophoresis. The proteins were then blotted onto nitrocellulose membrane using a Mini-Blot apparatus (e.g. Biorad), following manufacturers instructions. Immunodetection can then proceed, following the instructions of Western Breeze Chemiluminescent Detection Kit (Invitrogen), using a primary antibody prepared against purified recombinant GloxA protein (e.g. purified polyclonal rabbit IgG prepared by Institute of Veterinary and Medical Science, Adelaide, Australia). GloxA protein was detected in leaf cells of transgenic Arabidopsis expressing GloxA (FIG. 5) at levels of up to 0.8 ng/ug total protein.

Transgenic plant protein extracts were also assayed for GloxA activity by combining 100 μg of total protein (estimated 80 ng GloxA from Western Blot data) with 25 mM Tris-Cl pH7.2, 0.1 mM MgCl2. After preincubation at 37° C. for 2 minutes, 1 mM glyphosate was added to the reaction, which was then incubated with shaking for a further 60-120 minutes. After the required reaction time, reactions were derivatized with tosyl chloride and analysed by HPLC as described above. All enzyme assays were performed in triplicate and repeated at least twice to confirm data. Significant activity could be detected from leaves of T2 plants expressing GloxA mRNA (based on qPCR data) and protein (based on Western Blot data).

TABLE 9
GloxA activity in Arabidopsis plant cells expressing GloxA

| | GloxA Activity |
|---|---|
| Sample | (μmol/min/mg) |
| L-er (wildtype Arabidopsis) | 0 |
| A. thaliana GloxA T2#3 protein extract | 77.8 |
| A. thaliana GloxA T2#4 protein extract | 79.1 |
| A. thaliana GloxA T2#5 protein extract | 78.5 |
| L-er spiked with purified GloxA-E2 | 413.8 |
| Purified GloxA-E2 (positive control) | 497.5 |

### Example 7

### Production of Transgenic Maize Expressing GloxA

A chimeric gene comprising a cDNA encoding SEQ ID NO:1 in sense orientation with respect to the maize ubiquitin promoter (EP 342 926) that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (PciI and SmaI respectively) can be incorporated into the oligonucleotides used to amplify the cDNA to provide proper orientation of the DNA fragment when inserted into the digested vector pML103 (ATCC Accession No. 97366). Amplification is then performed in a standard PCR reaction. The amplified DNA is then digested with appropriate restriction enzymes PciI and SmaI and fractionated on an agarose gel. The appropriate band can be isolated from the gel and combined with the plasmid pML103. The DNA segment from pML103 contains a 1.05 kb SalI-NcoI promoter fragment of the maize 27 kD zein gene which is replaced, using standard techniques with the maize ubiquitin promoter, and a 0.96 kb SmaI-SalI fragment from the 3′ end of the maize 10 kD zein gene in the vector pGem9Zf(+) (Promega). Vector and insert DNA can be ligated at 15° C. overnight using standard procedures. The ligated DNA may then be used to transform E. coli XL1-Blue (Stratagene). Bacterial transformants can be screened by restriction enzyme digestion of plasmid DNA and limited nucleotide sequence analysis using the dideoxy chain termination method. The resulting plasmid construct would comprise a chimeric gene encoding, in the 5′ to 3′ direction, the maize ubiquitin zein promoter, a cDNA encoding GloxA, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al., 1975). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The particle bombardment method (Klein et al., 1987) maybe used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 304 of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains glyphosate (2 mg per liter). After an additional 2 weeks the tissue can be transferred to fresh N6 medium containing glyphosate. After 6 weeks, areas of about 1 cm in diameter of actively growing callus can be identified on some of the plates containing the glyphosate-supplemented medium. These calluses may continue to grow when sub-cultured on the selective medium:

Plants can be regenerated from the transgenic callus by first transferring clusters of tissue to N6 medium. After two weeks the tissue can be transferred to regeneration medium (Fromm et al., 1990).

### Example 8

### Production of Transgenic Soybean Expressing GloxA

An expression cassette composed of the cauliflower mosaic virus 35S promoter (Odell et al., 1985) and transcription terminator from the gene encoding the βsubunit of the seed storage protein phaseolin from the bean Phaseolus vulgaris can be used for expression of the instant enzymes in transformed soybean.

A cDNA fragment encoding an enzyme of the invention may be generated by polymerase chain reaction (PCR) using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the expression cassette.

Soybean embryos may then be transformed with the expression vector. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (U.S. Pat. No. 4,945,050). A DuPont Biolistic™ PDS1000/HE instrument (helium retrofit) can be used for these transformations.

A selectable marker gene which can be used to facilitate soybean transformation is a chimeric gene composed of the 35S promoter from Cauliflower Mosaic Virus, the hygromycin phosphotransferase gene from plasmid pJR225 (from E. coli) and the 3′ region of the nopaline synthase gene from the T-DNA of the Ti plasmid of Agrobacterium tumefaciens. The expression cassette can be isolated as a restriction fragment. This fragment can then be inserted into a unique restriction site of the vector carrying the marker gene.

To 50 μL of a 60 mg/mL 1 μm gold particle suspension is added (in order: 5 DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

Plants are tested for ability to grow when exposed to glyphosate.

### Example 9

### Production of Transgenic Cotton Expressing GloxA

For production to GloxA in cotton, the coding sequence of a protein of the invention may be operably linked to the subterranean clover stunt virus promoter (S7; WO 96/06932). The chimeric gene is operably linked to a selectable marker gene and introduced into a T-DNA vector. Cotton plants are transformed using the Agrobacterium mediated transformation technique. Transgenic cotton lines are identified by exposing the candidate transformants to glyphosate.

### Example 10

### Production of Transgenic Wheat Expressing GloxA

In order to produce GloxA in wheat, the polynucleotide comprising a sequence of nucleotides as provided in SEQ ID NO:6 is sub-cloned into a pPlex vector (Schunmann et al., 2003) such that the subterranean clover stunt virus promoter is able to drive gene transcription in a wheat cell.

Transformation of wheat embryos from the cultivar Bobwhite 26 is performed according to the method of Pellegrineschi et al. (2002). To confirm that the plants that were produced contained the construct, PCR analysis is performed on genomic DNA extracted from leaves using a FastDNA® kit (BIO 101 Inc., Vista, Calif., USA) according to the suppliers instructions. The DNA is eluted into 100 μl sterile deionized water and 1 μl used in PCR.

Plants are tested for ability to grow when exposed to glyphosate.

### Example 11

### Production of Transgenic Canola Expressing GloxA

Seedlings of Brassica napus are established in 5 cm pots. They are grown in a growth chamber at 24° C., 16/8 hour photoperiod. After 2.5 weeks they are transplanted to 15 cm pots and grown in a growth chamber at 15/10° C. day/night temperature, 16/8 hour photoperiod.

Four terminal internodes from plants just prior to bolting or in the process of bolting but before flowering are removed and surfaced sterilized in 70% v/v ethanol for 1 minute, 2% w/v sodium hypochlorite for 20 minutes and rinsed 3 times with sterile deionized water. Stems with leaves attached could be refrigerated in moist plastic bags for up to 72 hours prior to sterilization. Six to seven stem segments are cut into 5 mm discs maintaining orientation of basal end.

Agrobacterium expressing SEQ ID NO:6 is grown overnight on a rotator at 24° C. in 2 mls of Luria Broth containing 50 mg/l kanamycin, 24 mg/l chloramphenicol and 100 mg/l spectinomycin. A 1:10 dilution is made giving approximately 9×10⁸ cells per ml. This is confirmed with optical density readings at 660 μm. The stem discs (explants) are inoculated with 1.0 ml of Agrobacterium and the excess is aspirated from the explants.

The explants are placed basal side down in petri plates containing 1/10× standard MS salts, B5 vitamins, 3% sucrose, 0.8% agar, pH 5.7, 1.0 mg/l 6-benzyladenine (BA). The plates are layered with 1.5 ml of media containing MS salts, B5 vitamins, 3% sucrose, pH 5.7, 4.0 mg/l p-chlorophenoxyacetic acid, 0.005 mg/i kinetin and covered with sterile filter paper.

Following a 2 to 3 day co-culture, the explants were transferred to deep dish petri plates containing MS salts, B5 vitamins, 3% sucrose, 0.8% agar, pH 5.7, 1 mg/l BA, 500 mg/l carbenicillin, 50 mg/l cefotaxime, 200 mg/l kanamycin or 175 mg/l gentamicin for selection. Seven explants are placed on each plate. After 3 weeks they are transferred to fresh media, 5 explants per plate. The explants are cultured in a growth room at 25° C., continuous light (Cool White).

Plants are tested for ability to grow when exposed to glyphosate.

### Example 12

### Production of Transgenic Barley Expressing GloxA

In order to produce GloxA in barley, the polynucleotide comprising a sequence of nucleotides as provided in SEQ ID NO:6 is sub-cloned into a pPlex vector (Schunmann et al., 2003) such that the subterranean clover stunt virus promoter is able to drive gene transcription in a barley cell.

Transformation of barley embryos is performed according to the method generally as described by Pellegrineschi et al. (2002). To confirm that the plants that were produced contained the construct, PCR analysis is performed on genomic DNA extracted from leaves using a FastDNA® kit (BIO 101 Inc., Vista, Calif., USA) according to the suppliers instructions. The DNA is eluted into 100 μl sterile deionized water and 1 μl used in PCR.

Plants are tested for ability to grow when exposed to glyphosate.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

This application claims priority from U.S. 60/747,151, the entire contents of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abdullah et al. (1986) Biotechnology 4:1087.
Altschul et al. (1997) Nucl. Acids Res. 25: 3389-3402.
Balthazor and Hallas (1986) Appl. Environ Microbiol. 51:432-434.
Blair-Keith et al. (2001) Weed Sci. 49:375-380.
Capecchi (1980) Cell 22:479-488.
Castle et al. (2004) Science 304:1151-1154.
Cheng et al. (1996) Plant Cell Rep. 15:653-657.
Chu et al. (1975) Sci. Sin. Peking 18:659-668.
Clapp (1993) Clin. Perinatol. 20:155-168.
Cole et al. (2005) Nucleic Acids Res 33(Database Issue):D294-D296.
Curiel et al. (1992) Hum. Gen. Ther. 3:147-154.
Eglitis et al. (1988) Biotechniques 6:608-614.
Fitzgibbon and Braymer (1990) Appl. Environ. Microbiol. 56:3382-3388.
Fromm et al. (1990) Bio/Technology 8:833-883.
Futamata et al. (2005) Appl. Environ. Microbiol. 71: 904-911.
Fujimura et al. (1985) Plant Tissue Culture Letters 2:74.
Gleave (1992) Plant Mol. Biol. 20:1203-1207.
Gordon et al. (1999). Chemical-Biological Interactions 14:463-470.
Graham et al. (1973) Virology 54:536-539.
Grant et al. (1995) Plant Cell Rep. 15:254-258.
Harayama (1998). Trends Biotechnol 16:76-82.
Hayes et al. (2000) FEMS Microbiol. Lett. 186:171-175.
Jacob et al. (1985) J. Biol. Chem. 10: 5899-5905.
Jacob et al. (1988) Appl. Environ. Microbiol. 54:2953-2958.
Jander et al. (2003) Plant Physiology 131:139-146.
Kent-Moore et al. (1983) Appl. Environ. Microbiol. 46: 316-320.
Kishore and Jacob (1987) J. Biol. Chem. 262:12164-12168.
Klein et al. (1987) Nature 327:70-73.
Koziel et al. (1996) Plant Mol. Biol. 32:393-405.
Kryskol-Lupikca et al. (1997) Appl. Microbiol. Biotechnol. 48:549-552.
LeJuene et al. (1998). Nature 395:27-28.
Liu et al. (1991) Appl. Environ. Microbiol. 57:1799-1804.
Lu et al. (1993) J. Exp. Med. 178:2089-2096.
Liu et al. (2001) Appl. Environ. Microbiol. 67 (2):696-701.
Malik et al. (1989) Biofactors 2:17-25.
McGinnis and Madden (2004) Nucl. Acids Res. 32:W20-25.
McGrath et al. (1995) Eur. J. Biochem. 234:225-230.
McGrath et al. (1998) Appl. Environ. Microbiol. 64:356-358.
Metcalf and Wanner (1993) J. Bacteriol. 175: 3430-3442.
Mortl et al. (2004) J. Biol. Chem. 279: 29718-29727.
Needleman and Wunsch (1970) J. Mol. Biol. 48:443-453.
Objoska et al. (1999) Appl. Microbiol. Biotechnol. 51:872-876.
Odell et al. (1985) Nature 313:810-812.
Padgette et al. (1996) New Weed Control Opportunities: Development of Roundup Ready™ Gene. In Duke, S. O., ed., Herbicide-resistant crops, agricultural, environmental, economic, regulatory, and technical aspects. CRC Press Inc., Boca Raton, Fla., USA. pp. 53-84.
Pellegrineschi et al. (2002) Genome 45:421-430.
Petrikovics et al. (2000a). Toxicology Science 57: 16-21.
Petrikovics et al. (2000b). Drug Delivery 7: 83-89.
Pipke et al. (1988) Appl. Environ. Microbiol. 54: 1293-1296.
Schunmann et al. (2003) Functional Plant Biology 30:453-460.
Shinabarger and Braymer (1986) J. Bacteriol. 168:702-707.
Siehl et al. (2005) Pest. Manag. Sci. 61: 235-240.
Ternan et al. (1998a) World J. Microbiol. Biotechnol. 14: 635-647.
Ternan et al. (1998b) Appl. Environ. Microbiol. 64: 2291-2294.
Tomita et al. (1991) J. Chromatogr 566:239-243.
Toriyama et al. (1986) Theor. Appl. Genet. 205:34.
Vasil (1996) Phosphinothricin-resistant crops. In Duke, S. O., ed., Herbicide-resistant crops, agricultural, environmental, economic, regulatory, and technical aspects. CRC Press Inc., Boca Raton, Fla., USA. pp. 85-93.
Wackett et al. (1987) J. Bacteriol. 169: 710-717.
Wanner (1994) Phosphate regulated genes for the utilization o phosphonates in the members of the family Enterobacteriaceae. In: Phosphate in Microorganisms. Eds.
Torrigiani-Gorini, A., Yagil, E. and Silver, S. pp. 13-21. ASM press. Washington D. C.
Wagner et al. (1992) Proc. Natl. Acad. Sci. USA 89:6099-6103.
White and Metcalf (2004) J. Bacteriol. 186: 4730-4739.
Yakoleva et al. (1998) Appl. Microbiol. Biotechnol. 49: 573-578.

1. A substantially purified polypeptide that has one or more of the following features;
i) cleaves glyphosate to produce glycine,
ii) cleaves the phosphonomethyl C-3 carbon to nitrogen bond of glyphosate,
iii) has a greater efficiency for cleaving glyphosate than GOX (SEQ ID NO:3),
iv) has a specific activity for the cleavage of glyphosate which is greater than 550 μmolmin⁻¹ mg⁻¹, and/or
v) cleaves an amine-containing herbicide and comprises a sequence selected from:
a) an amino acid sequence as provided in SEQ ID NO:1, and
b) an amino acid sequence which is at least 25% identical to a).

2-14. (canceled)

15. The polypeptide according to claim 1 which is fused to at least one other polypeptide.

16. An isolated polynucleotide, the polynucleotide comprising nucleotides having a sequence selected from:
i) SEQ ID NO:2,
ii) a sequence of nucleotides encoding a polypeptide according to claim 1
iii) a sequence of nucleotides which is at least 25% identical to i),
iv) a sequence of nucleotides which hybridizes to i) under low stringency conditions,
v) a sequence of nucleotides complementary to i) to iv).

17-19. (canceled)

20. A recombinant polynucleotide comprising a promoter that functions in a plant cell, operably linked to a structural DNA sequence that encodes a polypeptide according to claim 1, operably linked to a 3′ polyadenylation sequence that functions in the cell, wherein the promoter is heterologous with respect to the structural DNA sequence and capable of expressing the structural DNA sequence to enhance resistance of the cell to an amine-containing herbicide.

21. A vector comprising a polynucleotide according to claim 16.

22. (canceled)

23. A host cell comprising at least one polynucleotide according to claim 16.

24. (canceled)

25. A recombinant cell that cleaves glyphosate and produces glycine, and/or comprises an introduced polypeptide that cleaves the phosphonomethyl C-3 carbon to nitrogen bond of glyphosate.

26. (canceled)

27. A process for preparing a polypeptide according to claim 1, the process comprising cultivating a host cell encoding said polypeptide, or a vector encoding said polypeptide, under conditions which allow expression of the polynucleotide encoding the polypeptide, and recovering the expressed polypeptide.

28. (canceled)

29. An isolated antibody which specifically binds a polypeptide according to claim 1.

30. A composition comprising at least one polypeptide according to claim 1, at least one polynucleotide encoding said polypeptide, a vector comprising said polynucleotide, a host and/or recombinant cell producing said polypeptide and/or an antibody which binds said polypeptide, and one or more acceptable carriers.

31-33. (canceled)

34. (canceled)

35. A method for selecting a recombinant cell, the method comprising
i) contacting a cell or a population of cells with a polynucleotide encoding a polypeptide according to claim 1 under conditions which allow uptake of the polynucleotide by the cell(s), and
ii) selecting a recombinant cell by exposing the cells from step i), or progeny cells thereof, to an amine-containing herbicide.

36-42. (canceled)

43. A method for cleaving an amine-containing herbicide, the method comprising contacting an amine-containing herbicide with a polypeptide according to claim 1.

44. (canceled)

45. A transgenic plant comprising an exogenous polynucleotide, the polynucleotide encoding at least one polypeptide according to claim 1.

46-47. (canceled)

48. A method of producing plants with enhanced resistance to an amine-containing herbicide comprising the steps of:
a) inserting into the genome of a plant cell a polynucleotide comprising: a promoter that functions in plant cells to cause the production of a RNA sequence, operably linked to a structural DNA sequence that causes the production of a RNA sequence that encodes a polypeptide according to claim 1, operably linked to a 3′ non-translated region that functions in plant cells to cause the addition of polyadenyl nucleotides at the 3′ end of the RNA sequence; where the promoter is heterologous with respect to the structural DNA sequence and adapted to cause sufficient expression of the polypeptide to enhance resistance to an amine-containing herbicide of a plant cell transformed with the DNA molecule;
b) obtaining a transformed plant cell; and
c) regenerating from the transformed plant cell a genetically transformed plant which has increased resistance to an amine-containing herbicide.

49. (canceled)

50. A method for cleaving an amine-containing herbicide in a sample, the method comprising exposing the sample to a transgenic plant producing a polypeptide according to claim 1.

51. (canceled)

52. A transgenic non-human animal comprising an exogenous polynucleotide, the polynucleotide encoding at least one polypeptide according to claim 1.

53. An isolated strain of Arthrobacter sp deposited under accession number V06/010960 on 11 Apr. 2006 at the National Measurement Institute, Australia.

54. A composition for cleaving an amine-containing herbicide, the composition comprising the strain of claim 53, and one or more acceptable carriers.

55. An extract of a host cell, a recombinant cell, a transgenic plant, a transgenic non-human animal, or an isolated strain of Arthrobacter sp, comprising a polypeptide according to claim 1.

56. A composition for cleaving an amine-containing herbicide, the composition comprising the extract of claim 55, and one or more acceptable carriers.

57. A method for cleaving an amine-containing herbicide, the method comprising exposing an amine-containing herbicide to the strain of claim 53 and/or an extract thereof.

58. An isolated bacterium which produces a polypeptide according to claim 1.

59. (canceled)

60. Use of an isolated naturally occurring bacterium which produces a polypeptide according to claim 1 for cleaving an amine-containing herbicide.

61. A polymeric sponge or foam for cleaving an amine-containing herbicide, the foam or sponge comprising a polypeptide according to claim 1 immobilized on a polymeric porous support.

62. A method for cleaving an amine-containing herbicide, the method comprising exposing an amine-containing herbicide to a sponge or foam of claim 61.

63. (canceled)

64. A part of a transgenic plant comprising a polypeptide according to claim 1.

65. (canceled)

66. A method of producing a polypeptide with enhanced ability to cleave an amine-containing herbicide, the method comprising
(i) altering one or more amino acids of a first polypeptide according to claim 1,
(ii) determining the ability of the altered polypeptide obtained from step (i) to cleave an amine-containing herbicide, and
(iii) selecting an altered polypeptide with enhanced ability to cleave an amine-containing herbicide, when compared to the first polypeptide.

67. (canceled)

68. A method for screening for a microorganism capable of cleaving an amine-containing herbicide, the method comprising
i) culturing a candidate microorganism in the presence of an amine-containing herbicide as a sole nitrogen source, and
ii) determining whether the microorganism is capable of growth and/or division.

69. (canceled)


SEQUENCE LISTING (9 sequences)

SEQ ID NO: 1 - protein, 472 aa, Arthrobacter sp. TBD
VKTTVFERNVPQTAVVSHALAGAAHRPYWIDHLQDSATRYPQLDGDAAADLVIVGGGYTGLWTALRAKERDPGRTVILLEGEQVAWAASGRNGGFCEASLTHGHENGKNRWPDELETLDRLGMENLDRMQEAVEKYAMDCEWERTGELNVAVEPHQVAWLQEDDTPGSVFLDQDAVRAEVNSPTYLAGRWHKDSVAMVHPYKLGLELARVATELGVVIYEGTRVRELNDHEHGVTVVTERGIVEAGHVVLGTNVFPSLLKRNQLMTVPVYDYALMTEPLTAEQLESIGWGKRQGIGDVANQFHYYRITKDHRILFGGYDALYFWGRRVDQKHEHQPATWEKIASHFFTTFPQLEGLKFTHKWAGPIDSSTQFCAFFGTARKGRVAYAAGFTGLGVGATAFAADVLLDLLAGLDTERTRLEMVRKRPLPFPPEPLASLGINLTRWSMDRADHNQGKRNIILKALDAVGLGFDS

SEQ ID NO: 2 - DNA, 1419 bp, Arthrobacter sp. TBD
gtgaagaccaccgttttcgaacggaatgtcccgcagaccgccgtcgtatcccatgcgctggcaggtgcggcccaccggccgtactggatcgaccacctccaggactcagccacccgctatccccaactcgacggcgatgctgccgcggacttagtgatcgtcggcggcggctacaccggattgtggacagcgttacgcgccaaggaacgcgaccccggcaggacagtgatcctgctggaaggggagcaggtggcttgggcagcttcgggccgtaatggtggtttctgcgaggcgagcctcacccacggccatgagaacggaaagaaccggtggccggacgagttggaaaccctggaccgcttgggcatggaaaacctggaccgcatgcaggaggccgtggagaagtacgccatggattgcgagtgggagcgcaccggtgagctcaacgttgcagtggaaccgcatcaagtggcgtggctccaggaggacgatacccccggcagcgtcttcctggaccaggacgccgttcgtgctgaggtgaactcgccaacttacctggcaggccgctggcacaaagactccgtggccatggtccatccgtacaaactaggcctggaattggcacgggtggctactgagttgggtgtggtgatttacgaaggcacacgagttcgcgagttgaacgatcatgagcacggcgtcacggtggtcacggagcgcggcatcgtggaagcgggacacgtggtccttggtaccaacgtgtttccttcacttctgaagcggaaccagctcatgaccgtcccggtgtacgactacgcactcatgaccgagcccctcaccgcagagcagttggagtccatcggttgggggaagcggcagggcatcggggacgtagccaaccagttccactattaccgcatcaccaaggaccaccggatcttgttcggaggctacgacgcgctctacttctggggacgccgcgttgaccaaaagcacgaacaccagccggcaacgtgggagaagatcgcctcgcacttcttcaccacgttcccgcagctcgaaggcctgaagttcacccataagtgggccggacccatcgattcgagcacccagttctgcgcgttcttcggcaccgcgcggaaggggcgggtggcatacgcggccggtttcacagggctgggagtgggagccacggccttcgccgccgatgtcctcctggacctgttggccggcctcgacaccgagcgcacccggctggagatggtacggaaacgccccctgcccttcccgcctgagcccctggcctcccttggcatcaacctgacgcgctggtccatggaccgggccgaccacaatcagggcaaaaggaacatcatcctcaaagccctcgacgccgtgggcctgggtttcgactcctga

SEQ ID NO: 3 - protein, 430 aa, Pseudomonas sp. LBAA
MSENHKKVGIAGAGIVGVCTALMLQRRGFKVTLIDPNPPGEGASFGNAGCFNGSSVVPMSMPGNLTSVPKWLLDPMGRCQSGSAISNHHAWLIRFLLAGRPNKVKEQAKALRNLIKSTVPLIKSLAEEADASHLIRHEGHLTVYRGEADFAKDRGGWELRRLNGVRTQILSADALRDFDPNLSHAFTKGILIEENGHTINPQGLVTLLFRRFIANGGEFVSARVIGFETEGRALKGITTTNGVLAVDAAVVAAGAHSKSLANSLGDDIPLDTERGYHIVIANPEAAPRIPTTDASGKFIATPMEMGLRVAGTVEFAGLTAAPNWKRAHVLYTHARKLLPALAPASSEERYSKWMGFRPSIPDSLPVIGRATRTPDVIYAFGHGHLGMTGAPMTATLVSELLAGEKTSIDISPFAPNRFGIGKSKQTGPAS

SEQ ID NO: 4 - DNA, 25 bp, Artificial
  Oligonucleotide primer
atggctgagaaccacaaaaaagtag

SEQ ID NO: 5 - DNA, 25 bp, Artificial
  Oligonucleotide primer
ttaacttgccggacccgtttgcttg

SEQ ID NO: 6 - DNA, 1445 bp, Artificial
  GloxA coding sequence optimized for expression in plants, includes added cloning sites at 5′ and 3′ ends as well as AACA just before start codon
ggtacctcgaggacaaatgaaaactactgtgttcgagagaaacgttcctcagactgctgttgtttctcatgctcttgctggtgctgctcatagaccttactggatcgatcacctccaagattctgctactagataccctcagctcgatggtgatgctgctgctgatcttgttatcgtgggtggaggatatactggactttggactgctctcagagctaaagagagagatcctggaagaactgtgatccttcttgagggtgaacaagttgcttgggctgcttctggaagaaatggtggattctgcgaggcttctctcactcatggacacgagaacggaaagaacagatggcctgatgagcttgagactctcgatagactcggaatggaaaacctcgatagaatgcaagaggctgtggagaagtacgctatggattgtgagtgggagagaactggtgaacttaacgttgctgtggagcctcatcaagttgcatggctccaagaggatgatactcctggatctgtgttccttgatcaggatgctgttagagctgaggtgaactctcctacttaccttgctggaagatggcataaggattctgtggctatggtgcatccttacaagcttggactcgaacttgctagagtggctactgagcttggagttgtgatctacgagggaactagagtgagagagcttaacgatcacgagcatggtgttactgttgtgactgagagaggaatcgttgaggctggacatgttgttcttggaactaacgtgttcccttctctccttaagagaaaccagctcatgactgtgcctgtttacgattacgctctcatgactgagcctcttactgctgaacagcttgagtctatcggatggggaaagagacaaggtatcggagatgtggctaaccagttccactactacagaatcactaaggatcacagaatcctcttcggaggatacgatgctctttacttctggggaagaagagttgatcagaagcacgagcatcaacctgctacttgggagaagatcgcttctcacttcttcactactttccctcagcttgagggacttaagttcactcataagtgggctggacctatcgattcttctactcagttctgcgctttcttcggaactgctagaaagggaagagttgcttacgctgctggattcactggacttggagttggagctactgctttcgctgctgatgttcttcttgatctcctcgctggacttgatactgagagaactagacttgagatggtgagaaagagacctcttccttttcctcctgagcctcttgcttctcttggtatcaacctcactagatggtctatggatagagctgatcacaaccagggaaagagaaacatcatcctcaaggctcttgatgctgttggactcggattcgattcttaatctagagctc

SEQ ID NO: 7 - DNA, 1419 bp, Artificial
  GloxA coding sequence optimized for expression in E. coli
atgaaaaccaccgtgtttgaacgtaacgtgccgcagaccgcggttgttagccatgcgctggccggtgcggcgcatcgtccgtattggattgatcatctgcaggatagcgcgacccgttatccgcagctggatggcgatgcggcagcggatctggtgattgtgggcggtggctataccggtctgtggaccgcgctgcgtgcgaaagaacgtgatccgggccgtaccgtgattctgctggaaggcgaacaggttgcgtgggcggcgagcggtcgtaacggcggcttttgcgaagcgagcctgacccatggccatgaaaacggcaaaaaccgttggccggatgagctggaaaccctggatcgtctgggcatggaaaatctggatcgtatgcaggaagcggttgaaaaatacgcgatggattgcgaatgggaacgtaccggcgagctgaacgtggcggtggaaccgcatcaggtggcgtggctgcaggaagatgataccccgggcagcgtgtttctggatcaggatgcggtgcgtgcggaagtgaacagcccgacctatctggccggtcgttggcataaagatagcgtggcgatggtgcatccgtataaactgggcctggagctggcccgtgtggcgaccgagctgggcgtggtgatttatgaaggcacccgtgtgcgtgagctgaacgatcatgaacatggcgtgaccgtggtgaccgaacgtggcattgtggaagcgggccatgtggtgctgggcaccaacgtgtttccgagcctgctgaaacgtaaccagctgatgaccgtgccggtgtatgattatgcgctgatgaccgaaccgctgaccgcggaacagctggaaagcattggctggggcaaacgtcagggcattggcgatgtggcgaaccagtttcattattatcgcatcaccaaagatcatcgtattctgtttggcggctatgatgcgctgtatttttggggccgtcgtgtggatcagaaacatgaacatcagccggcgacctgggaaaaaattgcgagccatttctttaccaccttcccgcagctggaaggcctgaaatttacccataaatgggcgggtccgattgatagcagcacccagttttgcgcgttttttggcaccgcgcgtaaaggccgtgttgcgtatgcggcgggttttaccggcctgggtgtgggtgcgaccgcgtttgcggcggatgtgctgctggatctgctggccggcctggataccgaacgtacccgtctggaaatggtgcgtaaacgtccgctgccgtttccgccggaaccgctggccagcctgggcattaacctgacccgttggagcatggatcgtgcggatcataaccagggcaaacgtaacattattctgaaagcgctggatgcggtgggcctgggctttgatagctaa

SEQ ID NO: 8 - protein, 473 aa, Brevibacterium linens BL2
MDVTAALADASTVPFWLDSDQRPEPLPPLTRDLEADLVIVGGGFTGLWTALQAKERDPGRHVVLVEANSIGWAASGRNGGFCAASLTHGYENGESHLPAENERLAQLGLDNLNEIEAAVTRYGMDVEFERTGELDVATEDYQVPELREAHDPEAGFIFYDQQELAQIVKSPTYKGALWDSREVAMVNPAKLCWELLRVIRELGVEVFEGTKVTDVSDRKTNVQITTLVTSQALEDCASAPCSIITAKRVALATNVFPSLLKRVSLHTVPVYDYALMTEPLSDEQLTSIGWDGRQGIGDCASRFHYYRLSADNRILFGGWDAVYHYGRQVSWKYDQRAETFETLAAHFYETFPQLQGVKFTHKWGGPIDTCSRFFSFFTTAYGKKVAMAAGFTGLGVGASRFAGTVMLDLLSGQDTELTQLEMVRKLPLPFPPEPVAWLGIQMTTNALIKADGNEGRRGPLLKALDAVGMGFDS

SEQ ID NO: 9 - protein, 472 aa, Arthrobacter aurescens TC1
MNTTVFERNVPQAAVVSRALAGAAHRPFWIDDLNDSATRYPQLGGDAAADLVIVGGGYTGLWTALRAKERDPGRTVILLEGERVAWAASGRNGGFCEASLTHGHENGRNRWPEELEILERLGMENLDRMQEAVEKYGMDCEWERTGELNVAVEPHQVSWLQEDDSPGSVFLDQEAVRAEVNSPTYLAGRWHKDSVAMVHPYKLGLELARVATELGVVIHEGTRVRELKDHEQGVTVVTERGNVEAGHVVLGTNVFPSLLKRNQLMTVPVYDYALMTEPLTAEQLESIGWGKRQGIGDVANQFHYYRITKDHRILFGGYDALYFWGRRVDQRHEHQPATWEKIASHFFTTFPQLEGLKFTHKWAGPIDSSTQFCAFFGTARKGRVAYAAGFTGLGVGATAFAADVLLDLLAGLDTERTRVEMVRKRPLPFPPEPLASLGINLTRWSMDRADHNQGKRNLILKALDAVGLGFDS